(19) United States
(12) Patent Application Publication
(10) Pub. No.: US 2022/0064238 A1
(43) Pub. Date: Mar. 3, 2022

(54) Methods And Compositions For Modifying Transcription Factor Activity by Targeting The Human Mediator Complex Using Cell Penetrating Memetic Peptides And Methods of Treating Cancer Using The Same

(71) Applicants: The Regents of the University of Colorado, a body corporate, Denver, CO (US); Yale University, New Haven, CT (US)

(72) Inventors: Dylan Taatjes, Boulder, CO (US); Alanna Schepartz Shrader, Berkeley, CA (US)

(21) Appl. No.: 17/464,469

(22) Filed: Sep. 1, 2021

Related U.S. Application Data
(60) Provisional application No. 63/073,245, filed on Sep. 1, 2020.

(51) Int. Cl. C07K 14/47

(52) U.S. Cl. CPC C07K 14/4703; C07K 2319/70; C07K 2317/31

(57) ABSTRACT

The invention includes novel systems, methods, and compositions to inhibit TF function by disrupting the TF-Mediator interaction. In this preferred aspect, functional mimics of TF activation domains may be rationally designed to block TF-Mediator binding and selectively inhibited TF-dependent transcription and may further incorporate a penta-arg motif for enhanced cell penetration. In one preferred embodiment of the invention novel stapled mimetic peptides of p53 activation domains incorporating a penta-arg motif were rationally designed to block p53 function by disrupting the p53-Mediator interaction. Additional aspects of the invention include methods of treating cancer in a subject, and other diseases related to the activity of the TFs, such as p53, and the Mediator complex, and its downstream TF-dependent transcription.

### CROSS REFERENCE TO RELATED APPLICATION

This U.S. Non-Provisional application claims the benefit of U.S. Application Ser. No. 63/073,245, filed Sep. 1, 2020, which is incorporated by reference in its entirety.

### GOVERNMENT INTEREST

This invention was made with government support under grant numbers CA170741 and GM117370 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 31, 2021, is named “90245-00561-Sequence-Listing-AF.txt” and is 4.94 Kbytes in size.

### TECHNICAL FIELD

The inventive technology relates to the field of transcription factor regulation and gene expression. In preferred embodiments, the inventive technology relates to novel systems, methods, and compositions to regulate gene expression function by disrupting the interaction of a transcription factor and the Mediator complex.

### BACKGROUND

Sequence-specific, DNA-binding transcription factors (TFs) drive a myriad physiological processes and their mutation or disruption underlies many human diseases. They are unquestionably high-impact targets for molecular therapeutics. Unfortunately, TFs have proven difficult to target with small molecules. Their DNA-binding domains are charged, and similar to other TFs their activation domains are typically unstructured and intrinsically disordered. Among the estimated ˜1600 TFs in the human genome, p53 stands out for its general importance in cancer biology.

Across many cell lineages, p53 functions as a tumor suppressor and can paradoxically function as an oncogene if it acquires specific “gain-of-function” mutations. Notably, p53 also plays key roles in mammalian development, aging, and stem cell biology. Like many TFs, the p53 protein possesses a DNA-binding domain and an activation domain (AD). The p53AD actually consists of two separate but closely-spaced domains, called AD1 (residues 14-26; SEQ ID NO. 1) and AD2 (residues 41-57; SEQ ID NO. 8). Whereas most transcriptional activation function can be attributed to p53AD1, loss-of-function p53AD1 mutations retain some ability to activate specific subsets of p53 target genes, and mutation of both AD1 and AD2 is required to mimic a p53-null phenotype.

The human Mediator complex contains 26 subunits and is generally required for RNA polymerase II (pol II) transcription. Mediator interacts extensively with the pol II enzyme and regulates its function in ways that remain poorly understood. However, a basic aspect of Mediator function is to enable TF-dependent activation of transcription. In fact, Mediator was originally discovered in S. cerevisiae using an in vitro assay to screen for factors required for TF-dependent transcription. Similar functions have been confirmed for mammalian Mediator. Because TFs do not bind pol II directly, it appears that they communicate their pol II regulatory functions indirectly, through the Mediator complex.

Upon binding Mediator, TFs induce conformational changes that remodel Mediator-pol II interactions to activate transcription. The p53 TF binds Mediator and this interaction has been shown to activate p53 target gene expression in vitro and in cells. Oncogenic mutations in p53AD1 disrupt p53-Mediator interactions and this correlates with loss of p53 function. Whereas specific residues and structural details remain unclear, the p53-Mediator interface appears to involve the MED17 subunit. Interestingly, other TFs (e.g. SREBP or nuclear receptors) activate transcription through interactions with different Mediator subunits.

Directly targeting TF activation domains has proven to be a difficult strategy to control TF function. As described below, the present invention sought to test whether the same outcome could be achieved by targeting Mediator instead. In one embodiment, p53 was selected as an exemplary embodiment because it is well-studied, biomedically important, and contains a well-characterized activation domain. An apparent obstacle was that Mediator is large (1.4 MDa, 26 subunits) and its p53 interaction site is not precisely defined. However, the present inventors reasoned that the p53 activation domain (residues 13-60) evolved to selectively interact with Mediator with high affinity. Consequently, the native p53AD structure and sequence were initially selected as a starting point, rather than screen thousands of drug-like compounds. To directly assess Mediator targeting, a defined in vitro transcription system that recapitulated p53- and Mediator-dependent transcription was employed.

### SUMMARY OF THE INVENTION

In one aspect the invention includes novel systems, methods, and compositions to inhibit TF function by disrupting the TF-Mediator interaction. In this preferred aspect, functional mimics of TF activation domains may be rationally designed to block TF-Mediator binding and selectively inhibited TF-dependent transcription. Functional mimics of TF activation domains may include mimetic peptides that competitively inhibit the interaction of the TF and Mediator complex. The mimetic peptides of the invention may further be modified to include hydrocarbon staples, which may confine and stabilize the mimetic peptide into a desired three-dimensional configuration. These functional mimics of TF activation domains may further be modified to increase cell penetration of the peptide into the cytosol and/or nucleus through the addition of a penta-arg motif (as described by Schepartz et al., in U.S. Pat. No. 10,227,384 which is incorporated herein by reference) that increases the function of an otherwise impermeant peptide.

In another aspect, functional mimics of TF activation domains may include bivalent mimetic peptides that competitively inhibit the interaction of the TF and the Mediator complex. The bivalent mimetic peptides of the invention may include separate domains coupled by a linker, and may further be modified to include hydrocarbon staples, which may modify one or more domains of the mimetic peptide into a desired three-dimensional configuration, such as an α-helical conformation. These bivalent mimetic peptides may further be modified to increase cell penetration of the peptide into the cytosol and/or nucleus through the addition of a penta-arg motif.

In another aspect, functional mimics of TF activation domains may include bivalent peptides that competitively inhibit the interaction of the TF and the Mediator complex. The bivalent peptides of the invention may include separate domains coupled by a linker, where, for example first domain is a mimetic peptide domain, and a second domain is a wild-type peptide domain. In the preferred aspect, the mimetic peptide domain may further be modified to include hydrocarbon staples, which may modify the mimetic peptide into a desired three-dimensional configuration, such as an α-helical conformation. One or more of the domains of the bivalent mimetic peptides may further be modified to increase cell penetration of the peptide into the cytosol and/or nucleus through the addition of a penta-arg motif.

In one aspect the invention includes novel systems, methods, and compositions to inhibit p53 function by disrupting the p53-Mediator interaction. In this preferred aspect, functional mimics of p53 activation domains may be rationally designed to competitively inhibit p53-Mediator binding and selectively inhibited p53-dependent transcription. These functional mimics of p53 activation domains may further be modified to increase cell penetration of the peptide into the cytosol and/or nucleus through the addition of a penta-arg motif.

In another aspect, functional mimics of p53 activation domains may include mimetic peptides of AD1 or AD2 that competitively inhibit the interaction of the p53 and the Mediator complex. The AD1 or AD2 mimetic peptides may further be modified to include hydrocarbon staples, which may modify the AD1 or AD2 mimetic peptide into a desired three-dimensional configuration, such as an α-helical conformation. The mimetic peptides of AD1 or AD2 may further be modified to increase cell penetration of the peptide into the cytosol and/or nucleus through the addition of a penta-arg motif.

In another aspect, functional mimics of p53 activation domains may include bivalent mimetic AD1 or AD2 peptides that competitively inhibit the interaction of the p53 and the Mediator complex. The bivalent AD1 or AD2 peptides of the invention may include separate activation domains coupled by a linker, and may further be modified to include hydrocarbon staples, which may modify the mimetic peptide into a desired three-dimensional configuration, such as an α-helical conformation. One or more of the domains of the bivalent mimetic peptides may further be modified to increase cell penetration of the peptide into the cytosol and/or nucleus through the addition of a penta-arg motif.

In another aspect, functional mimics of p53 activation domains may include bivalent peptides AD1 or AD2 that competitively inhibit the interaction of the p53 and the Mediator complex. The bivalent peptides of the invention may include separate domains coupled by a linker, where, for example, a first domain is a mimetic AD1 peptide domain, and a second domain is a wild-type AD2 peptide domain. In this preferred aspect, the mimetic peptide domain may further be modified to include hydrocarbon staples, which may modify the mimetic peptide into a desired three-dimensional configuration, such as an α-helical conformation. One or more of the domains of the bivalent mimetic peptides may further be modified to increase cell penetration of the peptide into the cytosol and/or nucleus through the addition of a penta-arg motif.

Another aspect of the invention includes methods of treating a disease or disorder, and preferably cancer by administering to a subject in need thereof a therapeutically effective amount of a functional mimic of one or more TF activation domains that has been rationally designed to block TF-Mediator binding and selectively inhibited TF-dependent transcription.

Another aspect of the invention includes methods of treating a disease or disorder, and preferably cancer associated with the activity of p53, by administering to a subject in need thereof a therapeutically effective amount of a functional mimic of one or more p53 activation domains that has been rationally designed to block p53-Mediator binding and selectively inhibited TF-dependent transcription.

Another aspect of the invention includes methods of treating a disease or disorder, and preferably cancer associated with the activity of p53, by administering to a subject in need thereof a therapeutically effective amount of a bivalent mimetic peptide of p53 activation domains AD1 and AD2 that has been rationally designed to block p53-Mediator binding and selectively inhibited TF-dependent transcription.

Another aspect of the invention includes methods of treating a disease or disorder, and preferably cancer associated with the activity of p53, by administering to a subject in need thereof a therapeutically effective amount of a bivalent peptide of p53 activation domains AD1 and AD2 wherein the AD1 domain is a memetic peptide and the AD2 domain is a wild-type activation domain, and wherein the that bivalent peptide has been rationally designed to block TF-Mediator binding and selectively inhibited p53-dependent transcription.

Another aspect of the invention includes methods of regulating, and more specifically downregulating the expression of p53-associated genes, by administering to a subject in need thereof a therapeutically effective amount of a bivalent peptide of p53 activation domains AD1 and AD2 wherein the AD1 domain is a memetic peptide and the AD2 domain is a wild-type activation domain, and wherein the bivalent peptide has been rationally designed to block TF-Mediator binding and selectively inhibited TF-dependent transcription.

Another aspect of the invention includes cell penetrating memetic peptide of the activation domain of a TF, having a penta-arg motif, that inhibits TF function by disrupting the TF-Mediator interaction. Another aspect of the invention stapled cell penetrating memetic peptide of the activation domain of a TF, having a penta-arg motif, that inhibits TF function by disrupting the TF-Mediator interaction. Additional aspect of this embodiment may include the generation of a library of activation domains of TFs that may rationally designed to block TF-Mediator binding and selectively inhibited TF-dependent transcription.

Another aspect of the invention includes isolated bivalent peptides according to SEQ ID NOs. 2-7, and 9-14.

Additional aims of the invention may include one or more of the following preferred embodiments:

### BRIEF DESCRIPTION OF THE FIGURES

FIGS. 1A-D. Human factors and peptides used for the reconstituted in vitro transcription assays. (A) Purified PIC factors. (B) WT p53AD1 sequence and sequences of p53AD1 peptides containing diverse penta-arg motifs. (C) WT p53AD2 sequence and sequences of p53AD2 peptides. (D) Residues Z and X represent a,a-disubstituted amino acids with olefin tethers for hydrocarbon-stapling. Residue B is an a,a-disubstituted amino acid with olefin tethers for hydrocarbon-stapling. Unstapled structures shown.

FIGS. 2A-D. In vitro transcription on chromatin templates reveals bivalent peptide is a potent and selective inhibitor of p53-dependent transcription activation. (A) IC₅₀ plot showing activity of bivalent peptide (n=3 to 8) vs. BP1.4 (stapled p53AD1 mimic; n=2 to 6) or a bivalent peptide with a mutated p53AD2 region (n=3 to 9). The reduced activity of BP1.4 and the AD2 mutant indicate that both p53 activation domains contribute to bivalent peptide function. (B) Representative data from experiments plotted in A. (C) IC₅₀ plot showing that bivalent peptide is selective for p53; repressive activity is reduced with GAL4-VP16 (n=2 to 6), which binds a different Mediator subunit compared with p53. (D) Representative data from experiments plotted in C. Vertical lines in plots represent standard error of the mean (panel A, C).

FIGS. 3A-E. Bivalent peptide blocks p53 response in Nutlin-treated HCT116 cells. (A) Simplified schematic showing Mediator recruitment and activation of pol II function via p53. (B) Structure of bivalent peptide. (C) GSEA hallmarks moustache plot showing robust p53 pathway activation upon treatment with Nutlin-3a (10 μM, 3 h). (D) GSEA hallmarks moustache plot showing effect of bivalent peptide in Nutlin-treated cells. Note the p53 pathway shows negative enrichment in cells treated with the bivalent peptide. (E) Heat map showing relative expression of core set of 103 p53 target genes in control (DMSO) vs. Nutlin-treated cells, in absence or presence of bivalent peptide. In agreement with GSEA plots from panels C & D, Nutlin-3a induction of p53 target genes is reduced in cells treated with bivalent peptide.

FIGS. 4A-E. Bivalent peptide blocks p53 pathway activation but has minimal effect on non-p53 target genes. (A, B) Representative IGV traces at select p53 target gene loci, showing induction with Nutlin-3a and reduced induction in presence of bivalent peptide (RNA-Seq experiment 1). (C, D) Representative IGV traces from RNA-Seq experiment 2, showing similar effects compared with RNASeq experiment 1. (E) Model; Mediator serves as a bridge between DNA-binding TFs and the pol II enzyme. The bivalent peptide effectively competes with p53 to prevent its binding to Mediator and subsequent activation of target genes in vitro and in cells. At non-p53 target genes, which are activated in part through other TF-Mediator interactions (via different Mediator subunits), the bivalent peptide has minimal impact on pol II transcription.

FIGS. 5A-C. In vitro screening protocol and stapled p53AD1 or p53AD2 peptides. (A) Promoter DNA template scheme. (B) Overview of reconstituted in vitro transcription assay (chromatin assembly optional; PIC=Pre-Initiation Complex: TFIIA, TFIIB, TFIID, TFIIE, TFIIF, TFIIH, Mediator, and pol II). (C) Representative in vitro transcription data from naked DNA templates, showing p53- and Mediator-dependence.

FIGS. 6A-D. Functional screening of stapled and unstapled p53AD1 mimics. (A) Representative data (in vitro transcription) using each peptide at 5 μM concentration, in presence (+) or absence (−) of GAL4-p53. Note that only BP1.4 and BP1.5 show ability to inhibit p53-activated transcription while not markedly affecting basal transcription. (B) Scatter plot summarizing in vitro transcription data for BP1.4 and BP1.5 peptides in absence (basal) or presence (activated) of GAL4-p53. (C) Representative data (top) and scatter plot (bottom) summarizing results from titration experiments with BP1.4 peptide under basal (−GAL4-p53) or activated (+GAL4-p53) conditions. (D) IC50 plot summarizing inhibitory activity of BP1.4 peptide. For all data panels (A-D), transcription was normalized to GAL4-p53 in absence of added peptide.

FIGS. 7A-C. Testing different PEG linker lengths to tether BP1.4 (stapled p53AD1 mimic) to p53AD2 sequence. (A) Schematic of 3 different PEG linkers to generate bivalent peptide 1, 2, or 3. (B) Representative in vitro transcription data and (C) scatter plot summary (n=4; bars=s.e.m.) of bivalent peptides with different PEG linker lengths. Note that a PEG6 or PEG10 linker showed enhanced ability to block p53-activated transcription, whereas PEG2 linker (i.e. bivalent peptide 1) did not.

FIGS. 8A-C. Bivalent peptide blocks p53AD-Mediator binding. (A) Overview of binding assays used. A crude Mediator sample was isolated from HeLa NE with a GST-SREBP affinity column (Naar et al., 1999) prior to incubation with p53AD (2 μM)±bivalent peptide (5 μM) in one of the protocols (right). (B) Scatterplot (left) summarizing p53AD binding to Mediator in presence of bivalent peptide. The percent binding is relative to p53AD bound to Mediator in absence of added peptide; p53AD quantitation was normalized to total Mediator, as assessed by quantitation of MED15 signal (and/or MED1 in some cases; n=4 biological and 6 total replicates). Red or orange dots correspond to data from binding protocol shown in panel A. Representative data (western blot) shown at right. (C) Binding assay denoted by orange dot (panel A) was used to probe VP16-Mediator binding (n=2), which revealed that that VP16-Mediator binding is not inhibited by the bivalent peptide. Normalization of bound GST-VP16 to MED15 in fact showed a 1.67-fold increase in Mediator-bound VP16 in the +peptide experiments. Representative western blot shown. Asterisk: free GST.

FIGS. 9A-F. Principal Component Analysis for RNA-Seq experiment 1 & 2. (A) PCA plot for biological replicate RNA-Seq experiment 1. (B) PCA plot for biological triplicate RNA-Seq experiment 2. (C-E) Linear regression analysis further demonstrates bivalent peptide specificity for p53 pathway. Shown are linear fits comparing (C) Nutlin response (water+Nutlin/water+DMSO) and Nutlin+peptide response (peptide+Nutlin/peptide+DMSO) for the 103 p53 pathway genes; (D) identical to (C) except for RNA-Seq experiment 2. (E) Linear regression analysis on a random set of 100 genes with a similar range of FPKM values observed for the 103 p53 pathway genes. A list of these genes and their expression levels is provided in Table 3. Linear fit performed on comparison of water+DMSO vs. peptide+DMSO expression. (F) Identical to (E) except for RNA-Seq experiment 2. Outliers plotted outside visible axes are denoted as triangles with their coordinates labeled on the plots.

FIGS. 10A-E. Bivalent peptide blocks p53 response in Nutlin-treated HCT116 cells (RNA-Seq experiment 2). (A) Simplified schematic showing Mediator recruitment and activation of pol II function via p53. (B) Structure of bivalent peptide. (C) GSEA hallmarks moustache plot showing p53 pathway activation upon treatment with Nutlin-3a (10 μM, 3 h). (D) GSEA hallmarks moustache plot showing effect of bivalent peptide in Nutlin-treated cells. Note the p53 pathway shows negative enrichment in cells treated with the bivalent peptide. (E) Heat map showing relative expression of core set of 103 p53 target genes in control (DMSO) vs. Nutlin-treated cells, in absence or presence of bivalent peptide. In agreement with GSEA plots from panels C & D, Nutlin-3a induction of p53 target genes is reduced in cells treated with bivalent peptide.

FIGS. 11A-G. Bivalent peptide has minimal impact on HCT116 transcriptome in absence of p53 stimulation. Principal Component Analysis (PCA) for RNA-Seq experiment 1 (A) and RNA-Seq experiment 2 (B) for unstimulated HCT116 cells (i.e. DMSO) in the presence or absence of bivalent peptide. Clustering of DMSO and DMSO+peptide samples highlighted by enclosed circle. (C) GSEA hallmarks moustache plot comparing unstimulated cells (i.e. DMSO) in presence or absence of bivalent peptide (RNA-Seq experiment 1 and experiment 2 combined). The plot reveals limited gene expression changes compared with Nutlin-3a treated cells (e.g. FIG. 3C). (D, E) Heat maps showing relative expression of core p53 target genes in unstimulated (DMSO) HCT116 cells, in absence or presence of bivalent peptide. RNA-Seq experiment 1 (D) or RNA-Seq experiment 2 (E). (F, G) GSEA hallmarks moustache plots showing effect of Nutlin in the presence of bivalent peptide (RNASeq experiment 1 or 2, respectively). Note the p53 pathway is still induced by Nutlin in the presence of bivalent peptide, but the magnitude of the induction is reduced compared with Nutlin+no peptide experiments (e.g. GSEA plot in FIG. 3C or FIG. 10C).

FIG. 12. Mediator complex is the major binding target for p53 in human cells. The p53 activation domain (AD; residues 1-70 of human p53 protein) was used as an affinity column (immobilized onto gluathione resin, expressed as GST-p53AD) and incubated with HeLa nuclear extract. Bound proteins were identified by LC-MS/MS. The Mediator complex was the top hit, even when compared with other similarly sized, large protein complexes such as TFIID or chromatin remodeler complexes.

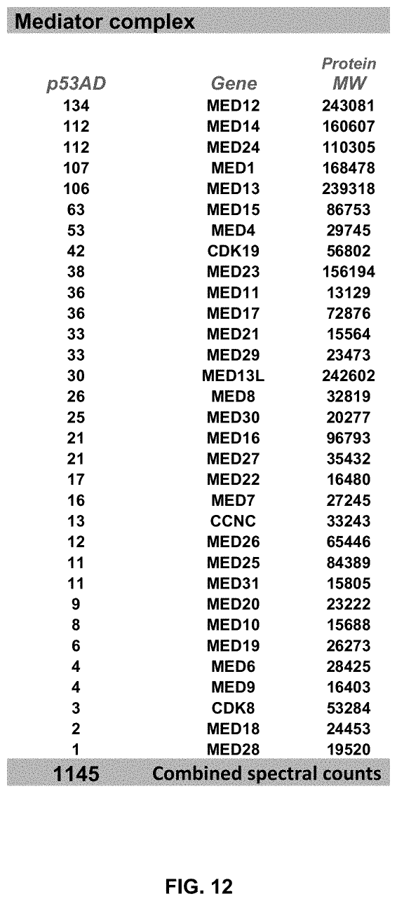

FIGS. 13A-B. Summary of ChIP experiments, showing that RNA polymerase II occupancy at the p53 target gene CDKN1A (p21) is reduced in cells treated with the bivalent peptide (peptide). A) Time course following Nutlin induction, to determine ideal time point to probe pol II occupancy at the p21 locus. At 3 h post-Nutlin, pol II occupancy is maximal, indicating that 3 h post-Nutlin is a good time point to probe for potential effects of the bivalent peptide. B) At 3 h post-Nutlin treatment, Nutlin has strongly induced pol II at the p21 locus, as expected. However, pol II occupancy is reduced in cells treated with the bivalent peptide. Because p53 binds Mediator to recruit pol II to gene promoters, these data further implicate the bivalent peptide in blocking stable p53-Mediator recruitment to p53 target genes. Pol II phosphorylated at serine-5 of its C-terminal domain was used for ChIP analysis, because this is a proxy for initiating pol II at gene promoters (start sites). Data represent biological triplicates. Similar results were observed genome-wide, using ChIP-seq, although the timing of induction across p53 target genes will vary (data not shown).

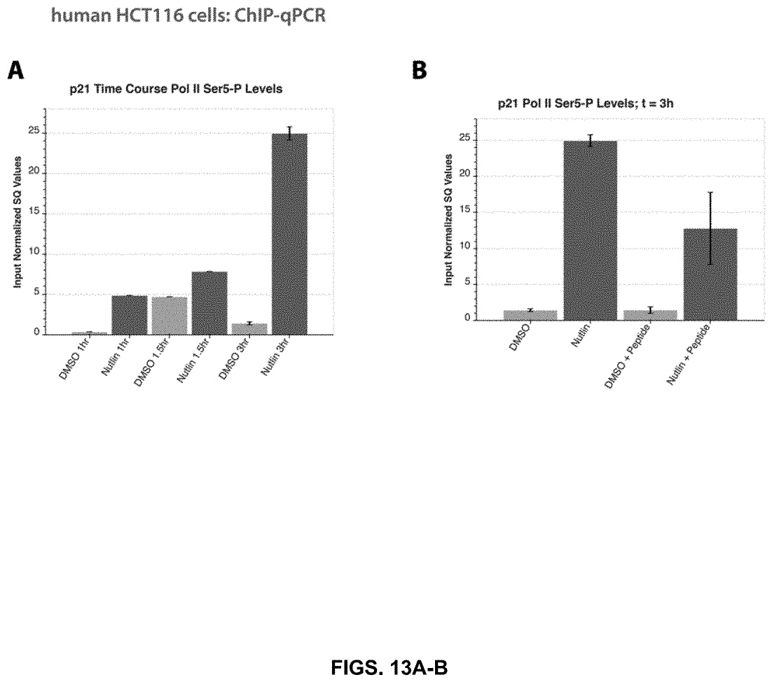

### DETAILED DESCRIPTION OF THE INVENTION

The invention includes novel systems, methods, and compositions to block TF function by disrupting the TF-Mediator interaction. In a preferred embodiment, functional mimics of TF activation domains may be rationally designed to block TF-Mediator binding and selectively inhibiting TF-dependent transcription. Functional mimics of TF activation domains may be configured to be monovalent, having a single TF activation domain that includes an amino acid sequence or protein domain that activates transcription when preferably tethered to a DNA-binding domain. Functional mimics of TF activation domains may also be configured to be multivalent peptides having a two or more TF activation domains that includes an amino acid sequence or protein domain that activates transcription when preferably tethered to a DNA-binding domain.

By targeting the TF-Mediator interface, the invention is able to produce a desired transcriptional outcome, while circumventing the need to directly inhibit TF, which as noted above, has proven to be a less than ideal strategy. Since TFs bind Mediator through different subunits, one additional embodiment of the invention includes the rational generation of libraries of TF activation domain mimics that may block TF-Mediator binding. Such strategy may further include the rational design of therapeutic compositions, which may preferably include TF activation domain mimics that may block TF-Mediator binding. Specific activation domains may be already known in the art or may be predicted from the sequence for known TFs and may form the basis for this rational design process. In this aspect, functional mimics may be generated from known FT activation domains, or from predicted FT activation domain sequences. Because such functional mimics of TF activation domains affect TF-driven transcription, such functional mimics may be incorporated into various assays, for example cell reporter assays or in vitro transcription assays. In this manner, the inventors may identify the activity of functional mimics of TF activation domains on downstream TF-driven transcription, and in particular the inhibitory effects of the invention's functional mimics on Mediator and on the resulting downstream TF-driven transcription.

The invention may include a peptide, and preferably a mimetic peptide configured to disrupt the interaction of a target TF and the Mediator complex. In one preferred embodiment, the invention may include a mimetic peptide comprising all or part of an activation domain of a TF, wherein said mimetic peptide competitively inhibits the interaction of the corresponding TF and the Mediator complex downregulate downstream TF driven gene expression. The TF activation domain of the mimetic peptide may be further configured to include stabilizing staples, such as a hydrocarbon staple linked between two residues on the peptide. In this manner, the hydrocarbon staple is configured to allow the activation domain of the mimetic peptide to form an α-helical conformation.

The mimetic peptide of a TF activation domain may be further configured to have enhanced cell and/or nuclear permeability. In this embodiment, the mimetic peptide may be modified to include a plurality of positively charged residues, such as lysine or arginine residues that may be recognized as a cellular or nuclear localization signal by a host cell. The TF activation domain of the mimetic peptide may specifically be modified to include a pent-arg motif to enhanced cell and/or nuclear permeability. The location of the positively charged residues, such as the arginine residues on the modified TF activation domain may be variably positioned.

The invention may further include a multivalent peptide, and preferably a bivalent peptide configured to disrupt the interaction of a TF and the Mediator complex. In one preferred embodiment, the invention may include a monovalent and/or a multivalent peptide, which may further preferably be a bivalent peptide; comprising a first activation domain of a DNA-binding transcription factor (TF) coupled with a second activation domain of said TF, wherein said bivalent peptide inhibits the interaction of the corresponding TF and the Mediator complex. One or both of the TF activation domains of the bivalent peptide may be further configured to include stabilizing staples. In one embodiment, the TF activation domains may be wild-type, or as described below, a memetic peptide having one or more hydrocarbon staples, and preferably a hydrocarbon staple linked between two residue on the peptide, that are configured to allow the TF activation domains of the bivalent peptide to form an α-helical conformation.

The TF activation domains of the bivalent peptide may be further configured to have enhanced cell and/or nuclear permeability. In this embodiment, one or both of the TF activation domains of the bivalent peptide may be modified to include a plurality of positively charged residues, such as a lysine or arginine that may be recognized as a cellular or nuclear localization signal by a host cell. One or both of the TF activation domains of the bivalent peptide may specifically be modified to include a pent-arg motif which may enhance cell and/or nuclear permeability. The location of the positively charged residues, such as the arginine residues on the modified TF activation domain may be variably positioned.

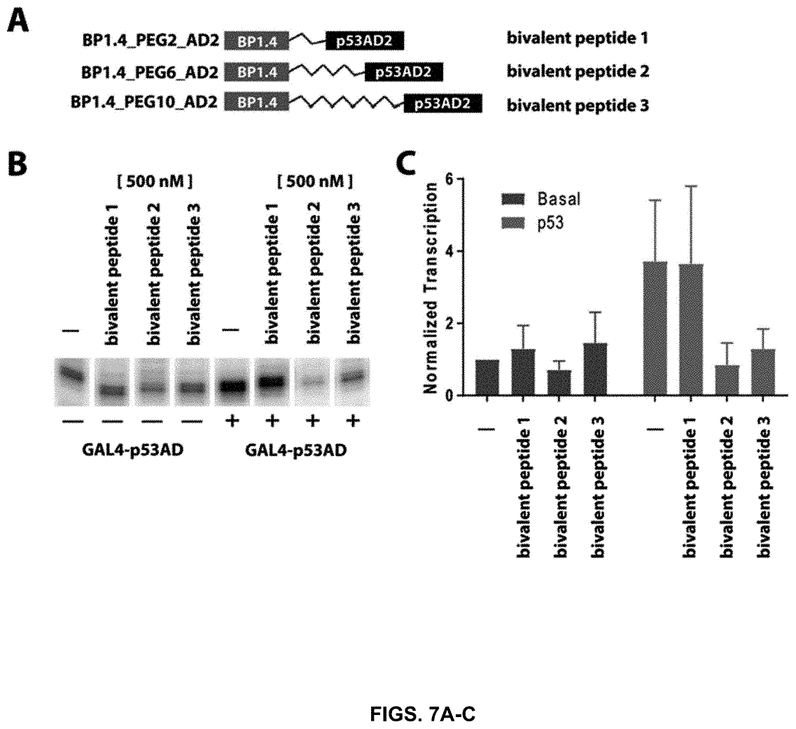

The invention may further include a bivalent peptide configured to inhibit the interaction of p53 and the Mediator complex and downregulates p53 target gene expression. In this embodiment, a bivalent peptide may include the first activation domain (AD1) of p53 coupled with the second activation domain (AD2) of p53 by a linker. As shown in FIG. 7A, AD1 (SEQ ID NO. 1) and AD2 (SEQ ID NO. 8) of p53 may be coupled with a PEG linker, having in this exemplary embodiment between 2-10 units, although alternative embodiments may include a linker having 1 PEG unit, as well as a linker having more than 10 PEG units.

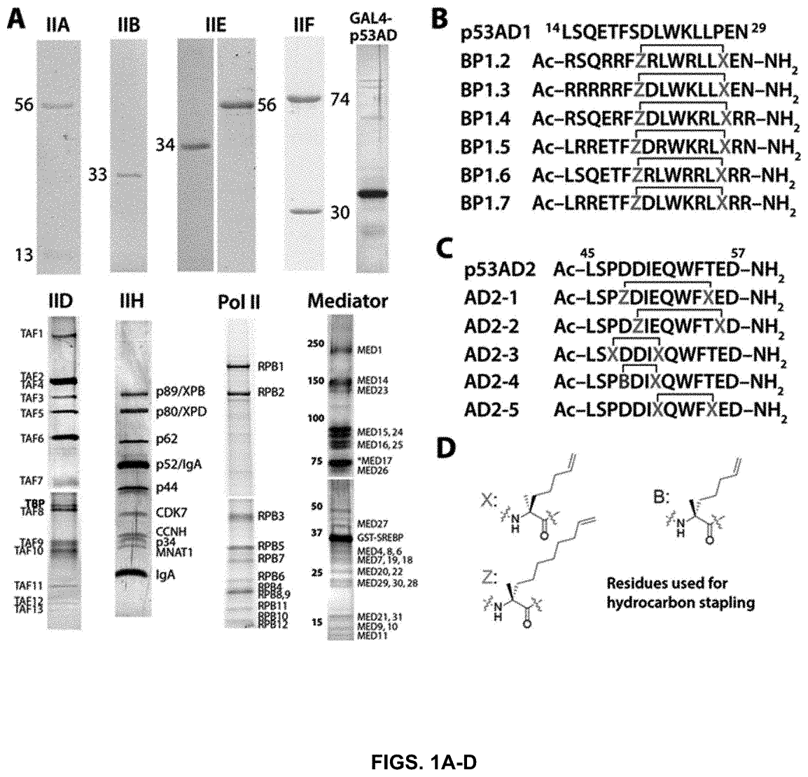

One or both of the TF activation domains may be modified to be mimetic peptides. In a preferred embodiment, AD1 or AD2 may be a wild-type sequence (SEQ ID NO. 1 and 8), or alternatively AD1 or AD2 may be mimetic peptides configured to inhibit the interaction of p53 and the Mediator complex. As noted above, AD1 or AD2 may further incorporate one or more staples that may stabilize the individual peptide domains in a three-dimensional configuration. As shown in FIG. 1E, in one embodiment, the AD1 peptide may be a mimetic AD1 peptide and may further include a hydrocarbon staple, forming a stapled mimetic AD1 peptide. As also shown in FIGS. 1E-D, the hydrocarbon staple on AD1 or AD2 may include a hydrocarbon staple positioned between residues Z, X and/or B, wherein Z, X and/or B are a,a-disubstituted amino acids with olefin tethers for hydrocarbon-stapling as generally shown in FIG. 1B. In one specific example shown in FIG. 1E, the hydrocarbon staple on hydrocarbon stapled mimetic AD1 peptide (SEQ ID NO. 2-7) may include a hydrocarbon staple between position i, and i+7—which correspond to residues 20 and 27 of AD1 respectively. Naturally, the variable positioning of the hydrocarbon staple is exemplary only and may in some embodiments include a hydrocarbon staple between position i, and between i+3 to i+7, among other examples.

One or both of the p53 activation domains may be modified to enhance cellular penetration and/or nuclear localization. As shown in FIG. 1E, in one example AD1 may be modified to include a plurality of substitute positively charged residues, in this case arginine residues. In this embodiment, the substituted arginine residues generate a penta-arg motif that enhances cellular penetration and/or nuclear localization.

In one preferred embodiment, the invention may include a multivalent peptide having a first and second TF activation domain coupled with a linker. In this embodiment, one or more of the domains may include a mimetic peptide incorporating a hydrocarbon staple and a penta-arg motif, while the other domains may include wild-type activation domains of a TF, wherein the multivalent peptide is configured to inhibit TF function by disrupting the TF-Mediator interaction.

In a specific example of this embodiment, the invention may include a bivalent peptide having a first AD1 domain coupled with a second AD2 domain by a linker. In this embodiment, the first AD1 domain may be a mimetic AD1 peptide incorporating a hydrocarbon staple and a penta-arg motif (SEQ ID NO. 2-7), while the second AD2 domain may include a wild-type AD2 (SEQ ID NO. 8), or optionally an AD2 domain incorporating a hydrocarbon staple and a penta-arg motif. In a preferred embodiment, a bivalent peptide of the invention may include a first AD1 domain, which may be a mimetic AD1 peptide incorporating a hydrocarbon staple and a penta-arg motif (SEQ ID NO. 2-7), while the second AD2 domain may include wild-type AD2 domain (SEQ ID NO. 8). This memetic bivalent peptide (SEQ ID NO. 14) competitively inhibits p53 function by disrupting the p53-Mediator interaction and inhibit downstream expression of p53 target genes.

As noted above, the ability to inhibit the interaction of a TF and the Mediator complex may be useful in treating a number of disease and disorders. In particular, the ability of the compositions of the invention to disrupt the p53-Mediator interaction and inhibiting downstream expression of p53 target genes may be an effective therapeutic approach to treating cancer. As such, the invention further includes novel systems, methods, and compositions to block p53 function by disrupting the p53-Mediator interaction. In this preferred aspect, rational design and activity-based screening may be used to characterize a stapled peptide, with functional mimics of both p53 activation domains AD1 (SEQ ID NO. 1) and AD2 (SEQ ID NO. 8), that blocked p53-Mediator binding and selectively inhibits p53-dependent transcription in vitro and in human cancer cells.

Additional embodiments of the invention include novel methods of inhibiting the expression of one or more genes regulated by a target TF in a cell, comprising the step of introducing to a cell a therapeutically effective amount of a peptide, and preferably a bivalent memetic peptide of the invention, wherein the peptide, and preferably a bivalent memetic peptide inhibits TF-Mediator binding and selectively inhibits TF-dependent transcription in vitro and in human cells, and preferably cancer cells.

In one specific embodiment, the invention includes novel methods of inhibiting the expression of one or more p53 target genes in a cell, comprising the step of introducing to a cell a therapeutically effective amount of a peptide, and preferably a bivalent memetic peptide of the invention, such as the bivalent peptide according to SEQ ID NO. 14, wherein the bivalent peptide inhibits p53-Mediator binding and selectively inhibits p53-dependent transcription in vitro and in human cells, and preferably cancer cells characterized by activity of p53. In this example, p53 target genes that may be downregulated in response to the inhibition of the p53-Mediator complex may include, but not be limited to the group consisting of: KLHL30, MDM2, ACER2, BTG2, SESN1, TP53INP1, GPR87, FAS, CDKN1A, PHLDA3, PRDM1, FAM212B, GRIN2C, SESN2, DDB2, IKBIP, ANKRA2, FBXO22, ZNF79, DRAM1, PRKAB1, PLK3, ZNF337, POLH, TNFRSF10B, DCP1B, ABCA12, XPC, SUSD6, DDIT4, TIGAR, TMEM68, ZMAT3, RRM2B, GDF15, PPM1D, GADD45A, SERPINE1, BBC3, INPP1, ETV7, ASCC3, RPS27L, PTP4A1, ZNF561, NADSYN1, TGFA, CCNG1, AMZ2P1, ISCU, KANK3, TSKU, DGKA, GATS, LIF, ATF3, PRKX, BAX, RAP2B, PLK2, EFNB1, WDR63, CD82, TRIAP1, BLOC1S2, TMEM63B, TP53I3, EPS8L2, CSNK1G1, SULF2, CDC42SE1, PANK1, FDXR, ANXA4, PSTPIP2, RNF19B, AMZ2, DUSP14, PARD6G, APOBEC3H, FHL2, CDIP1, ZNF385A, BLCAP, CMBL, FAM210B, PNPO, ZNF561-AS1, E124, CYFIP2, LRP1, APOBEC3C, TRAF4, HES2, CEL, PGF, PHPT1, NINJ1, SERTAD1, ADIRF, NUPR1, CYSRT1, and KLHDC7A.

Additional embodiments of the invention include a method of treating a disease or disorder, and preferably cancer in a subject in need thereof, comprising the step of administering to the subject a therapeutically effective amount of a peptide and/or bivalent peptide, and preferably a bivalent peptide of the invention according to the amino acid sequence SEQ ID NO. 14, wherein the peptide, inhibits p53-Mediator binding and selectively inhibited TF-dependent transcription resulting in the treatment of the cancer in preferably a human subject, and preferably wherein the cancer is characterized by activity of p53.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest.

As used herein, the term “transcription factors” refers to proteins that are directly involved in regulation of transcription initiation through binding to regulatory elements, such as regulatory elements within promoter regions, or binding to other proteins for collective binding to thus allowing RNA polymerase to transcribe DNA. In one preferred embodiment, transcription factors may interact with the Mediator complex and drive transcription activity.

As used herein, the term “mimics,” “mimetics,” “peptide mimetics,” “mimetic peptide” or “mimetic proteins” refers to biologically active compounds that mimic the biological activity of a peptide or protein. In one example, mimetic peptide may refer to a portion of a peptide, such as an activation domain of a peptide that has substantially the same structural and functional characteristics of the polypeptides of the invention. As a result of this similar active site geometry, mimetic-peptide has effects on biological systems similar to the biological activity of the peptide. Peptide mimetics that are structurally similar to therapeutically useful peptides may be used to produce an equivalent or enhanced therapeutic or prophylactic effect. Generally, mimetic peptides are structurally similar to a paradigm polypeptide (i.e., a polypeptide that has a biological or pharmacological activity). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. A mimetic peptide also includes D-reverse peptides and enantiomers as defined below. The term “reverse D-peptide” refers to a biologically active protein or peptide consisting of D-amino acids arranged in an inverted order compared to the L-amino acid sequence of a peptide. Thus, the terminal carboxy residue of an L-amino acid peptide becomes the amino terminal for the D-amino acid peptide, and so on. For example, the peptide, ETESH, becomes HdSdEdTdEd, where Ed, Hd, Sd and Td are the D-amino acids corresponding to the L-amino acids E, H, S and T, respectively. The term “enantiomer” refers to a biologically active protein or peptide, wherein one or more of the L-amino acid residues in the amino acid sequence of a peptide are replaced by corresponding D-amino acid residues.

The term, “Mediator” or “Mediator complex” refers to a large complex of protein subunits that regulates RNA polymerase II transcription by transducing signals from transcription activators bound to enhancer regions to the transcription machinery, which is assembled at promoters as the preinitiation complex (PIC) to control transcription initiation.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma, and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one. The term “cancer” includes for example, lung, breast, stomach, pancreas, prostate, bladder, bone, ovary, skin, kidney tumors, face, colon, intestine, stomach, rectum, esophagus, blood, brain and its meninges, spinal cord and its meninges, muscles, connective tissues, adrenal, parathyroid, thyroid, uterus, testis, pituitary, reproductive organs, liver, gallbladder, eye, ear, nose, throat, tonsils, mouth, lymph nodes, lymphoid system, and other organs. The term “cancer” is also intended to encompass all forms of carcinomas, sarcomas, and human melanomas that occur in poorly differentiated, moderately differentiated, and well differentiated forms.

The term “staple” or staples peptide” or “hydrocarbon staple” as used herein refers to the intramolecular or intermolecular connection (also referred to as cross-linking) of two peptides or two peptide domains (e.g., two loops of a helical peptide). In various embodiments, the stapled peptide has 1, 2, or 3 staples, which may preferably be a hydrocarbon staple.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “coupled” when applied to a mimetic peptides, or linkers, refers to linkage by preferably covalent bonding. A coupled protein may include a “bivalent protein” which refers to a protein with two binding sites. A “bivalent protein” may include multiple homologous, or heterologous domains, and may also contain a linker. The term “linker” refers to a molecule or group of molecules that connects two molecules, such as TF activation domains, such as the AD1 or AD2 domains of p53, and serves to place the two molecules in a preferred configuration. As used herein, a “bivalent protein” may further encompass multi-valent peptides having 2 or more binding sites.

A “domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein. The function of a domain, such as an activation domain may modulate gene expression, for example through interaction with the Mediator complex.

As used herein, “inhibits,” “inhibition” refers to the decrease in protein activity relative to the normal wild type level, or control level. Inhibition may result in a decrease in protein activity, such as the interaction of a TF, such as p53, with the Mediator complex in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “inhibits,” “inhibition” may also refer to the changes in gene expression relative to the normal wild type level, or control level. Inhibition may result in a differential, and preferably decreased, gene expression, such as genes responsive to p53's interaction with the Mediator complex, in response a composition of the invention by less than 10%, 10%, 15%, 20%, 25%, 30%, 35%, 40%, 45%, 50%, 55%, 60%, 65%, 70%, 75%, 80%, 85%, 90%, 95% or 100%.

The term “competitive inhibition” as used herein refers to competition between the variant, such as a composition of the invention, and a substrate, such as the Mediator complex, for the enzyme or the substrate binding partner such as a TF, and preferably p53, for example, where competition for binding of the enzyme where only one can bind at a time.

By the term “modulating,” as used herein, is meant mediating a detectable increase or decrease in the level of a mRNA, polypeptide, or a response in a subject, or a cell or tissue of a subject, as compared with the level of a mRNA, polypeptide or a response in the subject, or a cell or tissue of the subject, in the absence of a treatment or compound, and/or compared with the level of a mRNA, polypeptide, or a response in an otherwise identical but untreated subject, or cell or tissue of the subject. The term encompasses perturbing and/or affecting a native signal or response thereby mediating a beneficial therapeutic response in a subject, preferably, a human.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “therapeutically effective amount” as used herein refers to that amount of a composition of the invention being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

As used herein, “subject” refers to a human or animal subject. In certain preferred embodiments, the subject is a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Administration of a composition of the invention, and preferably a bivalent composition of the invention, may be effected by any method that enables delivery of the compositions to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for example a composition of the invention, and preferably a bivalent composition of the invention, calculated to produce the desired therapeutic effect in association with the required pharmaceutical carrier. The specification for the dosage unit forms of the invention are dictated by and directly dependent on (a) the unique characteristics of the composition and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of a composition of the invention, and preferably a bivalent composition of the invention, administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

In one preferred embodiment, a therapeutically effective amount or dosage of a composition of the invention, and preferably a bivalent composition of the invention, may be a dosage sufficient to inhibit the interaction of a TH, such as p53, with the Mediator complex.

A “pharmaceutical composition” or “pharmaceutical composition of the invention” refers to a composition of the invention, and preferably a bivalent composition of the invention, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional anticancer therapeutic agent, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages.

Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single agents, for example a pharmaceutical composition of a composition of the invention, or a pharmaceutical composition of a bivalent memetic peptide of the invention, or may be administered in combination with other anti-cancer therapeutic agents, in particular standard of care agents appropriate for the particular cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis. Pharmaceutical compositions suitable for the delivery of compounds of the invention, such as a memetic peptide as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: In Vitro Assay to Test p53-Activated Vs. Basal Transcription

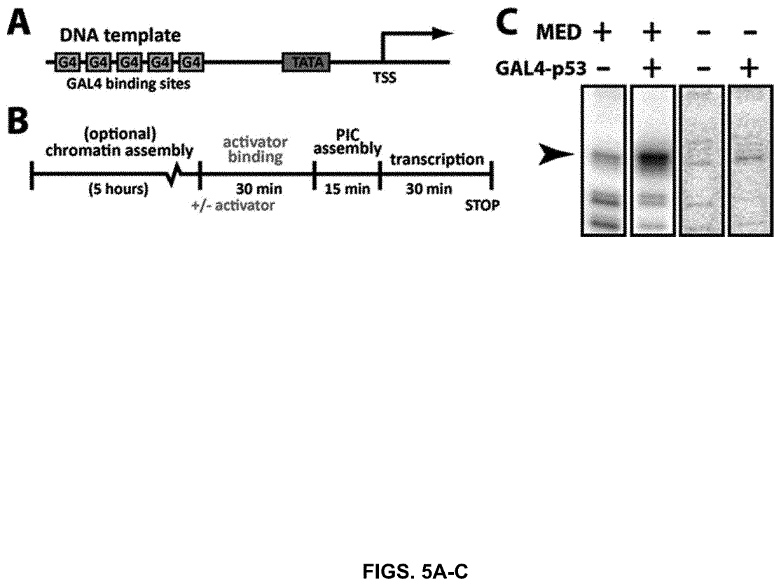

To screen peptides for the ability to selectively block p53-dependent transcription, we required an assay that enabled p53-dependent activation but that could also support basal (i.e. activator-independent) transcription. The present inventors had previously established an in vitro transcription assay (using highly purified human factors (FIG. 1A). A key feature of this assay was that both activated and basal transcription could be reconstituted on naked DNA templates (i.e. DNA templates not assembled into chromatin). To adapt this assay for purposes of measuring basal vs. p53-activated transcription, we generated templates with Gal4 DNA binding sites upstream of the adenovirus major late promoter sequence (FIG. 5A, B). Upon titration of a Gal4 DNA Binding Domain (DBD)-p53 Activation Domain (AD; residues 1-70) fusion protein into this system, we observed pol II-dependent transcription that was dependent on p53AD and Mediator (FIG. 5C). Reactions containing Gal4-p53AD generally produced about two- to four-fold more transcripts compared to reactions with no activator (FIG. 5C). Because experiments without Gal4-p53AD produced a low level of “basal” transcription that could be quantitated, this system allowed assessment of both p53-activated and basal transcription.

### Example 2: Design and Synthesis of Stapled Peptides

We designed hydrocarbon-stapled peptide mimetics of the AD1 (SEQ ID NO. 1) and AD2 (SEQ ID NO. 2) regions of p53. Hydrocarbon staples were employed to promote helicity within the peptides. Hydrocarbon-stapled peptides have previously been developed to mimic the α-helical portion of p53AD1 that binds MDM2/MDM4 with the goal of blocking the p53-MDM2/MDM4 interaction and restoring wild-type p53 activity. Indeed, ALRN6924, a hydrocarbon-stapled p53AD1 peptide mimetic developed from dual MDM2/MDM4 inhibitor ATSP-7041, has been evaluated in clinical trials (ClinicalTrials.gov identifier: NCT02264613 and NCT02909972). For the p53AD1 mimetics (SEQ ID NOs 2-7), we synthesized N-acetylated versions of the penta-arg-containing peptides BP1.2-BP1.7, which are based on residues 14-29 of p53 (FIG. 1). This panel of peptides contains an i, i+7 hydrocarbon staple at positions 20 and 27 and five arginine residues, which may be refer to as a penta-arg motif as described by Schepartz et al., in U.S. Pat. No. 10,227,384 which is incorporated herein by reference, grafted into various positions, which were originally introduced to improve the cell penetration or cytosolic access of the peptides. For the p53AD2 mimetics (SEQ ID NOs 9-13), we designed a panel of hydrocarbon-stapled peptides that varied the length and position of the hydrocarbon staple and spanned residues 45-57 of p53 (FIG. 1C). The panel included two peptides with an i, i+7 hydrocarbon staple (AD2-1 (SEQ ID NO. 9) and AD2-2 (SEQ ID NO. 10)), two peptides with an i, i+4 staple (AD2-3 (SEQ ID NO. 11) and AD2-4 (SEQ ID NO. 12)), and one peptide with an i, i+3 staple (AD2-5 (SEQ ID NO. 13)). Furthermore, both stapled and unstapled variants of the p53AD2 peptides were generated.

### Example 3: Functional Screening of Stapled Peptide Mimics of p53AD1 and p53AD2

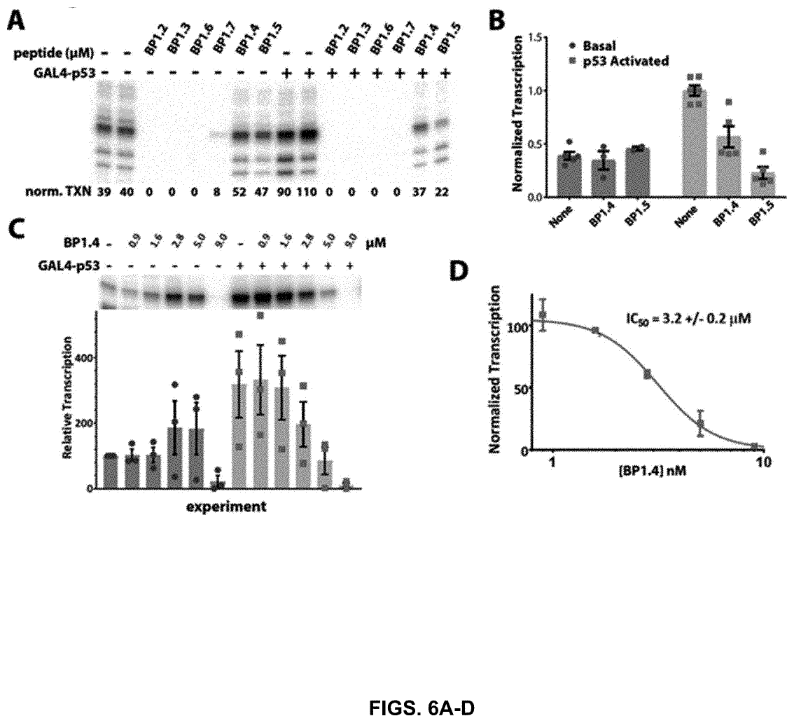

Starting with the stapled p53AD1 peptides, we tested whether any would block p53-dependent transcription activation without inhibiting basal transcription. Initial screens were completed with 5 μM of each peptide (BP1.2-BP1.7 (SEQ ID NOs 2-7, respectively); FIG. 1i). At this concentration, all peptides significantly reduced p53 activated transcription, but BP1.4 (SEQ ID NO. 4) and BP1.5 (SEQ ID NO. 5) did not affect basal transcription (FIG. 6A). In follow-up experiments, we observed that the BP1.5 peptide negatively affected basal transcription to some degree, whereas the BP1.4 peptide did not (FIG. 6B). That is, the BP1.4 peptide was most selective for blocking p53-dependent vs. basal transcription. We therefore chose the BP1.4 peptide for further testing.

To determine a concentration range in which the BP1.4 peptide selectively blocked p53-activated transcription but not basal transcription, we titrated BP1.4 into transcription reactions at concentrations between 0.9 μM and 9 μM (FIG. 6C). Interestingly, BP1.4 activated basal transcription at concentrations of 4 μM and above, which could reflect weak binding of BP1.4 to Mediator (i.e. mimicking p53AD) to promote transcription activation. Consistent with this result, promoter-bound pol II complexes are activated upon p53-Mediator binding in. Although basal transcription was inhibited at the 9 μM titration point, the weak BP1.4-dependent activation made the determination of the IC₅₀ for basal transcription impossible using an inhibitor response curve. The IC₅₀ describing the inhibition of p53-activated transcription by BP1.4 was 3.2±0.2 μM (FIG. 6D). The concentration window in which BP1.4 selectively blocked activated transcription was therefore relatively narrow.

We next tested the p53AD2 peptides (FIG. 1C) in a similar manner. In contrast to the p53AD1 peptides, the p53AD2 peptides either had no effect on p53-activated transcription or non-specifically inhibited both p53-activated and basal transcription at 5 μM (data not shown). Testing further at different peptide concentrations (i.e. increasing concentration if no activity was observed at 5 μM or decreasing concentration if both activated and basal transcription were inhibited) did not reveal any stapled p53AD2 peptides with specificity for p53-activated transcription. These results were not entirely unexpected, as p53AD2 appears to play a lesser role (vs. p53AD1) in activation of p53 target genes in.

### Example 4: A Bivalent Peptide Selectively Blocks p53-Dependent Activation In Vitro

We hypothesized that covalently linking two peptides with low-to-moderate affinity and specificity could generate a cooperatively binding “bivalent” peptide with improved ability to inhibit p53-dependent activation. Given the inactivity of the p53AD2 peptides tested, we elected to tether the BP1.4 peptide to the wild type p53AD2 sequence. In this way, we hoped to generate a competitive inhibitor of p53AD-Mediator binding by recapitulating the combined landscape of p53AD1/AD2 interactions. And because the p53AD1 portion was stapled (i.e. BP1.4), this “bivalent peptide” might effectively compete with WT p53 for Mediator binding.

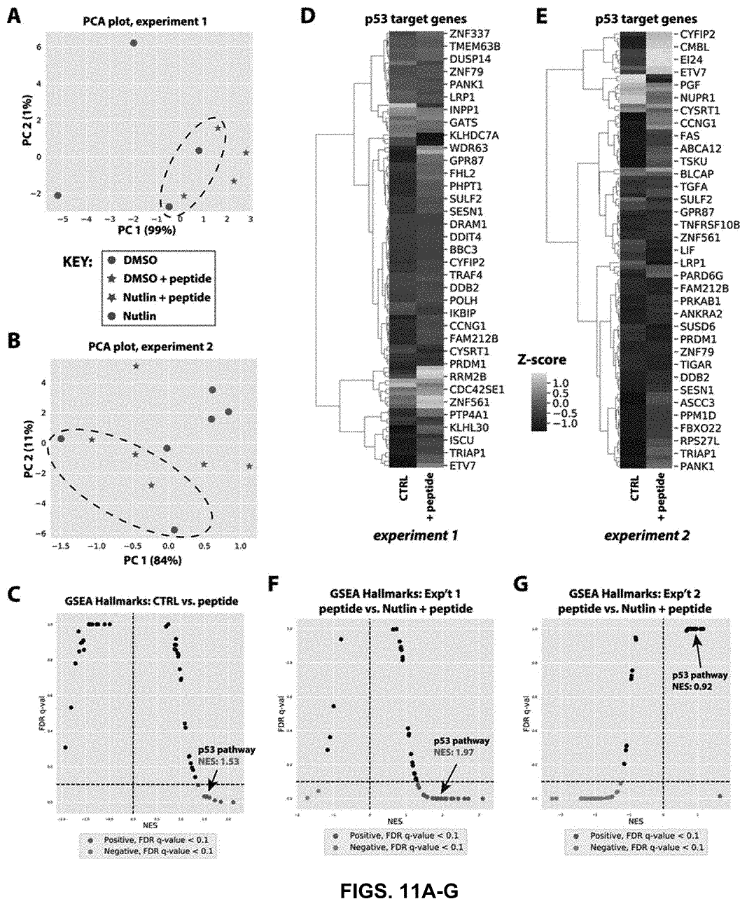

We synthesized and tested three bivalent peptides (BP1.4+p53AD2 sequence (SEQ ID NO. 15)) that contained a 2-, 6- or 10-unit polyethylene glycol (PEG) linker (bivalent peptide 1, 2, or 3; FIG. 7A). Notably, the bivalent peptides were significantly more potent inhibitors of p53-activated transcription than BP1.4 alone. As shown in FIG. 7B, bivalent peptides (500 nM) containing either a 6- or 10-unit PEG linker (i.e. bivalent peptide 2 or 3) inhibited p53-activated but not basal transcription. By contrast, the bivalent peptide with a 2-unit PEG linker (i.e. bivalent peptide 1) did not inhibit transcription at 500 nM (FIG. 11B). Because the bivalent peptide containing a 6-unit PEG linker (i.e. BP1.4_PEG6_p53AD2; bivalent peptide 2) was easier to synthesize (vs. 10-unit PEG), it was used for the experiments outlined below.

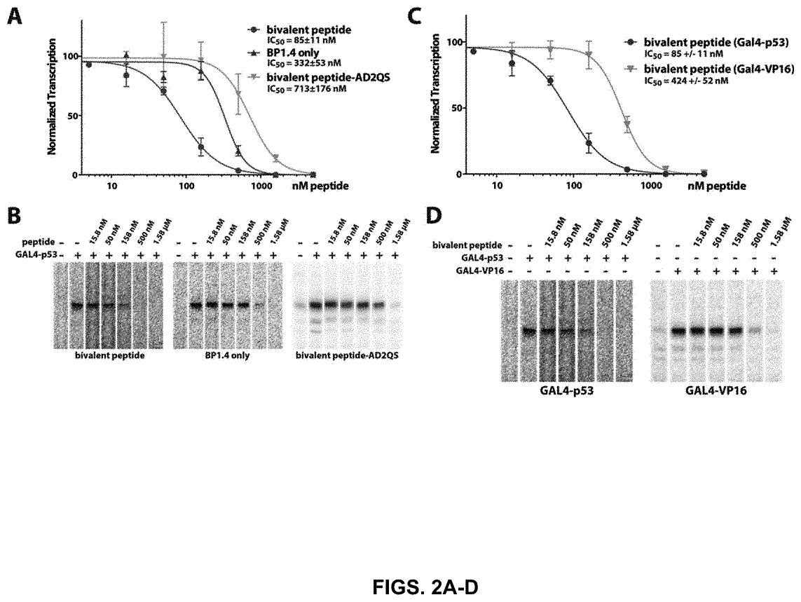

The in vitro transcription assays on naked DNA templates demonstrated improved potency of the bivalent peptide and also confirmed that it inhibited p53-activated transcription but not basal transcription. We next tested its function on more physiologically relevant chromatin templates, in which basal transcription is repressed. In fact, a TF activation domain (such as p53AD) and Mediator are required for transcription on chromatin templates, due to the ability of Mediator to relay the activation signal from the TF directly to the pol II enzyme. In vitro transcription assays with chromatin templates revealed that the bivalent peptide had a half maximal inhibitory concentration (IC₅₀) of 85 nM when added to reactions activated by Gal4-p53AD. By contrast, the BP1.4 peptide alone had an IC₅₀ of 330 nM in these assays (FIG. 2A, B). Upon introduction of a QS mutation into p53AD2, which blocks its activation function in vivo, the IC₅₀ increased to 713 nM, about eight-fold higher than the bivalent peptide and two-fold higher than BP1.4 alone (FIG. 2A, B). Collectively, these results suggest that both p53AD1 and p53AD2 contribute to Mediator-dependent transcriptional activation in vitro.

Finally, we assessed whether the bivalent peptide would selectively block p53-dependent transcription compared with VP16, a viral activation domain. Whereas p53 and VP16 both interact with Mediator, they do so through different subunits (MED17 and MED25, respectively). In contrast to Gal4-p53AD (85 nM), the bivalent peptide had an IC₅₀ of 424 nM in the presence of Gal4-VP16 (FIG. 2C, D). These data, which resulted from identical DNA templates assembled into chromatin, suggested that the bivalent peptide selectively blocked the p53-Mediator interaction vs. the VP16-Mediator interaction; this was further supported by biochemical provided below. The fact that VP16-dependent transcription was reduced at higher concentrations of bivalent peptide suggests transcriptional squelching may be occurring, in which high levels of TF activation domains repress transcription in vitro or in cells, presumably through competition for binding of co-activators such as Mediator.

### Example 5: A Bivalent Peptide Selectively Blocks p53-Dependent Activation In Vitro

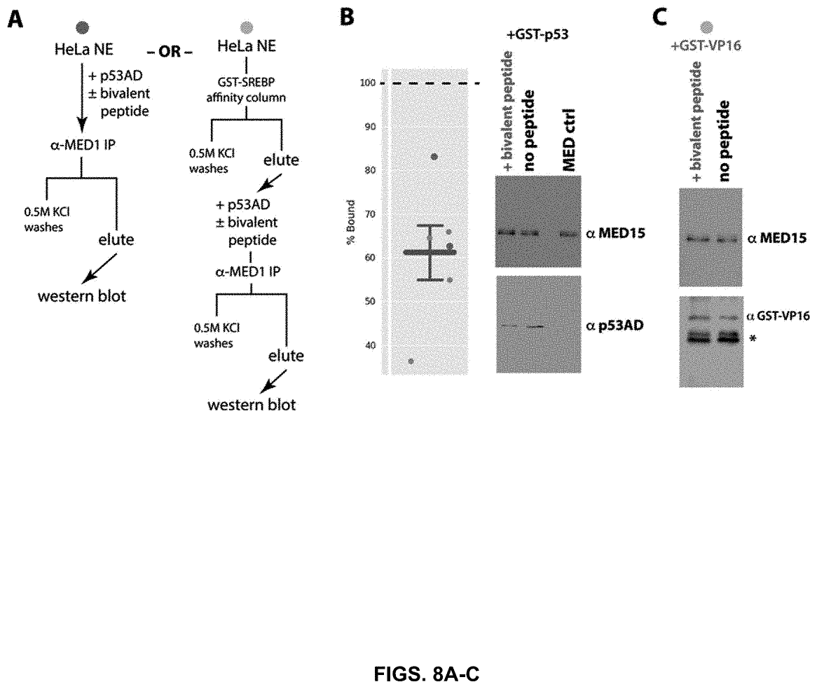

To further test whether the bivalent peptide would selectively block the p53AD-Mediator interaction, we performed a series of biochemical experiments, as outlined in FIG. 8A. The p53AD can bind Mediator with specificity and apparent high affinity. For example, the p53AD itself (residues 1-70) is sufficient to selectively isolate Mediator from partially purified cell extracts. As shown in FIG. 8B, p53AD binding to Mediator was markedly reduced (approximately 60% bound vs. no peptide controls) in the presence of the bivalent peptide; by contrast, the bivalent peptide did not reduce VP16AD binding to Mediator (FIG. 8C). These data are consistent with in vitro transcription results (FIG. 2) and reveal that the bivalent peptide functions, at least in part, by blocking p53AD-Mediator interactions.

By using purified factors, the in vitro data (FIG. 8 & FIG. 2) established direct inhibition of p53AD-Mediator binding by the bivalent peptide. ChTP assays were considered to further assess inhibition of Mediator recruitment in cells; however, limited available quantities of the bivalent peptide precluded a rigorous analysis. Because the genomic occupancy of Mediator correlates with pol II recruitment and transcription, we instead turned to RNA-Seq, which requires fewer cells and less optimization, to test bivalent peptide function in cells.

### Example 6: Bivalent Peptide Reduces Activation of p53 Targets in Nutlin-Stimulated HCT116 Cells

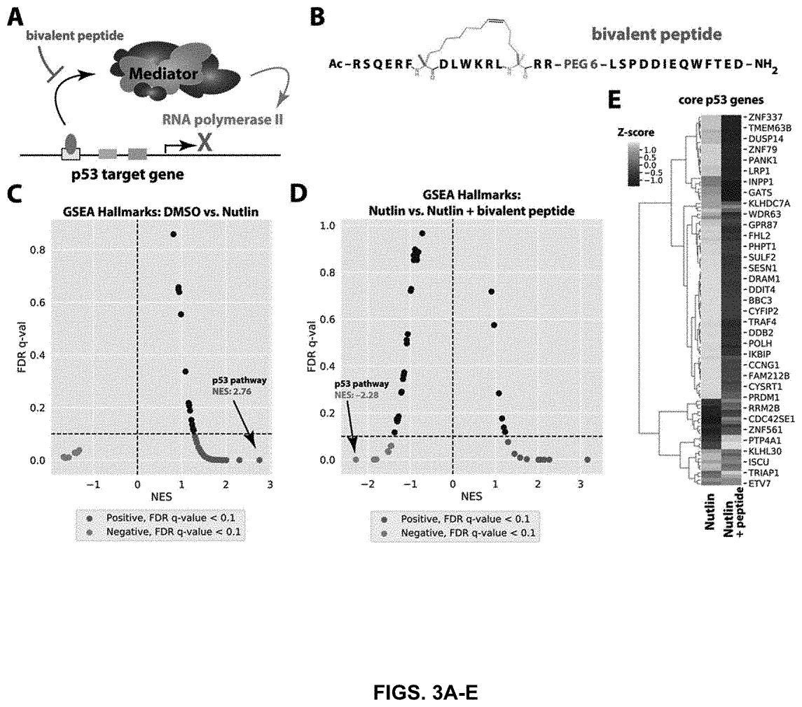

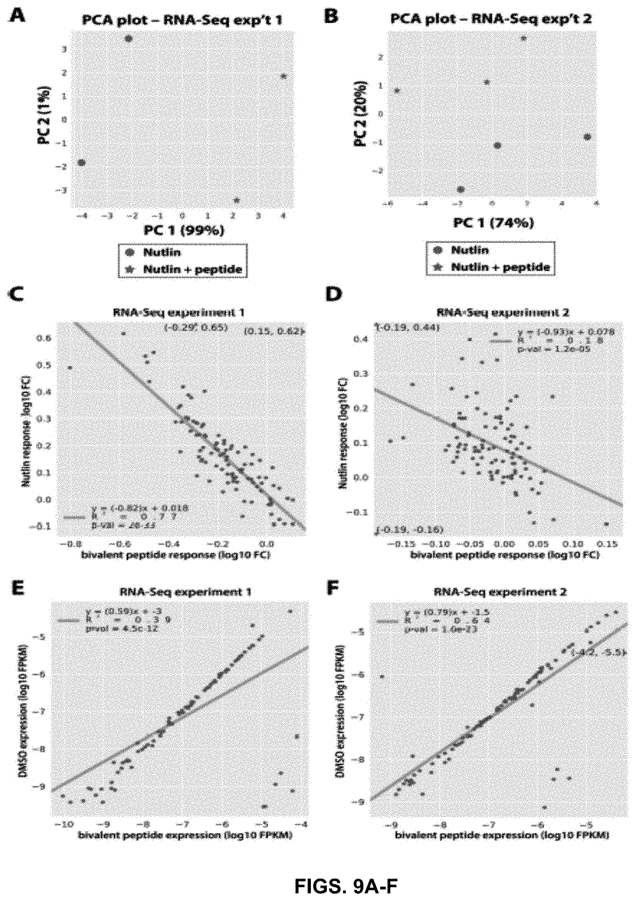

Prior analysis of the BP1.4 peptide showed that it is not effectively taken up by cells, and given its larger size, the bivalent peptide was expected to have poor cellular uptake. To circumvent this issue, we used a well-tested electroporation protocol to enhance cell uptake of the bivalent peptide (see Methods). HCT116 cells were evaluated either in the presence of bivalent peptide or vehicle (water), with or without Nutlin-3a (FIG. 3A, B). Nutlin-3a is a small molecule that activates and stabilizes p53 by inhibiting MDM2, a repressor of p53. A 3 h treatment time was used based upon experiments that suggested that the bivalent peptide was biologically active for only a limited time in cells (see Methods). After 3 h Nutlin-3a treatment (or DMSO control, ±bivalent peptide), nuclear RNA was isolated and biological replicate RNA-Seq libraries were prepared (FIG. 9A; Table 1).

As expected, Nutlin-3a induced expression of p53 target genes (FIG. 3C), consistent with previous studies in HCT116 cells. Strikingly, however, Nutlin-induced activation of p53 target genes was diminished in cells treated with the bivalent peptide (vs. controls; FIG. 3D). Inhibition by the bivalent peptide was observed across a core set of p53 target genes, identified as p53-inducible across cell types (FIG. 3E).

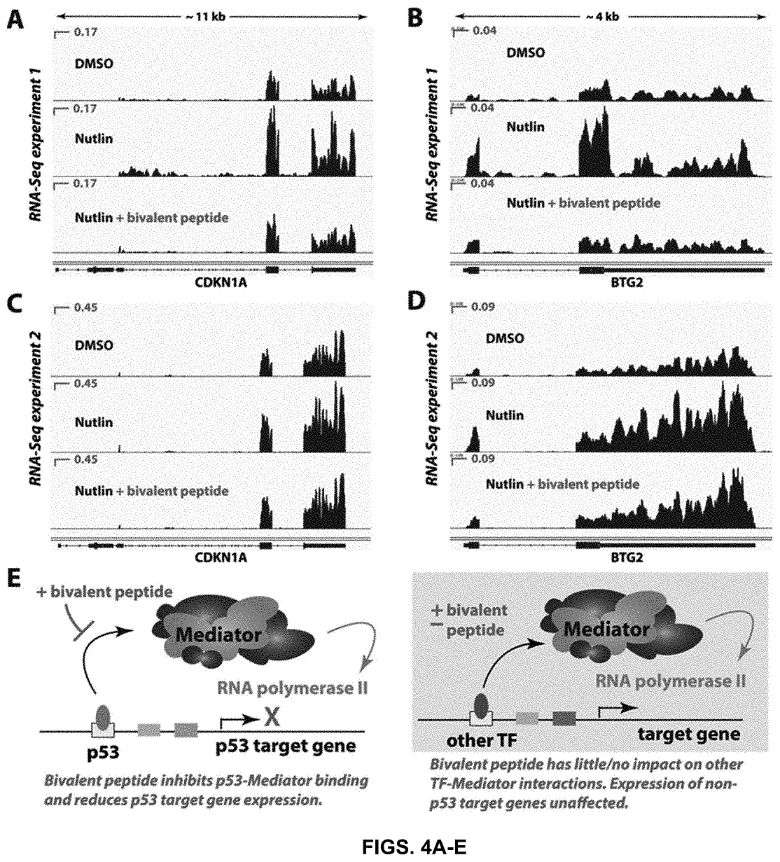

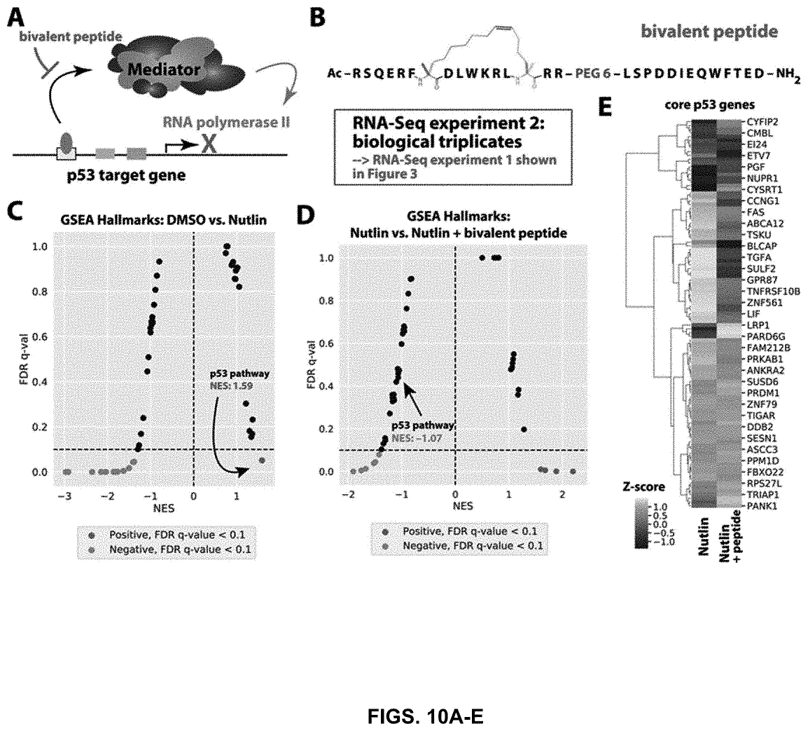

A confounding issue with these experiments was that cellular uptake of the bivalent peptide could not be accurately measured and could potentially vary from experiment-to-experiment. To assess this possibility more thoroughly, we completed an additional round of RNA-Seq experiments in control vs. Nutlin-treated HCT116 cells (FIG. 9B; Table 1). These additional experiments (RNA-Seq experiment 2) were completed with a slightly modified protocol that included treatment in serum-free media as an effort to enhance peptide uptake. Importantly, the data from RNA-Seq experiment 2 (biological triplicate; FIG. 10) were consistent with the first series of biological replicates (RNA-Seq experiment 1; FIG. 3C-E) and in each case, repression of p53-dependent transcription by the bivalent peptide tracked with Nutlin induction levels (FIG. 9CE). Nutlin-induced activation of p53 target genes was inhibited in the presence of the bivalent peptide, providing further evidence that the bivalent peptide blocks p53 response in cells. However, the scope and magnitude of the effect was reduced in this second, independent set of RNA-Seq experiments (e.g. compare FIG. 3C, D & FIG. 10C, D). This broadly consistent but variable magnitude effect can also be seen from RNA-Seq reads at specific p53 target genes (RNA-Seq experiment 1, FIG. 4A, B; RNA-Seq experiment 2, FIG. 4C, D). The overall p53 response to Nutlin-3a was reduced in RNA Seq experiment 2 (GSEA p53 pathway NES=2.76 in RNA-Seq experiment 1 vs. NES=1.59 in RNASeq experiment 2), which therefore reduced the transcriptional impact of the bivalent peptide. However, we cannot rule out variable cellular uptake of the bivalent peptide as a contributing factor. Taken together, the data from both sets of RNA-Seq experiments demonstrate 1) Nutlin induction of p53 target genes and 2) inhibition of p53 target gene activation in the presence of the bivalent peptide.

### Example 7: Bivalent Peptide has Modest Transcriptional Effect in Absence of p53 Activation

An expectation of our experimental strategy was that the bivalent peptide, which was designed based upon p53AD structure, would selectively block p53 function. Support for selective p53 inhibition was observed in vitro, upon comparison of the effects of the bivalent peptide on GAL4-p53AD vs. GAL4-VP16AD (FIG. 2C, D). RNA-Seq experiments in HCT116 cells represented a more rigorous test for selectivity. HCT116 cells express hundreds of sequence-specific, DNA-binding TFs, including high level expression of TFs that define the cell lineage. For HCT116 cells, these TFs include SREBF1, ELF3, JUNB, NR2F1, and MYC. To assess the general impact of the bivalent peptide on pol II transcription, we compared RNA-Seq data from DMSO control cells (i.e. no Nutlin treatment) in the presence/absence of bivalent peptide. The data revealed that, in contrast to Nutlin-treated cells, the bivalent peptide had a minor effect on pol II transcription, genome-wide, in both sets of RNA-Seq experiments. For example, PCA plots showed clustering of DMSO control and peptide-treated samples compared with Nutlin-treated samples (FIG. 11A, B), and GSEA showed a modest impact of the bivalent peptide on global transcription in the absence of Nutlin stimulation; however, enrichment of some hallmark gene sets was detected (FIG. 11C). Interestingly, evidence for p53 activation was observed in bivalent peptide-treated cells (vs. untreated controls) in RNA-Seq experiments 1 and 2, and modest p53 activation could be seen in heatmaps of p53 target genes (FIG. 11D, E). This observation could reflect an ability of the bivalent peptide to mimic p53AD-Mediator binding to activate transcription (as suggested in vitro for the BP1.4 peptide, FIG. 6C) and/or an ability to inhibit MDM2 and/or MDM4 binding to p53 in HCT116 cells. Overall, the RNA-Seq data from uninduced cells (i.e. not treated with Nutlin) were consistent with our in vitro results (FIG. 2) and suggest that the bivalent peptide is selective for p53 and does not inhibit other TF-Mediator interactions that would otherwise more broadly impact pol II transcription (FIG. 4E).

A final comparison that could be made from the RNA-Seq data was to assess the effect of Nutlin-3a in peptide-treated cells. Given that the bivalent peptide alone could trigger a mild p53 response (FIG. 11D, E), we asked whether Nutlin would still activate p53 target genes in this context. As expected, GSEA comparisons revealed that 1) Nutlin was able to induce p53 target gene expression even in the presence of the bivalent peptide, but 2) Nutlin induction of p53 target genes was reduced in this context (FIG. 11F, G). For instance, the Normalized Enrichment Score (NES) for Nutlin-induced p53 pathway activation in the absence of bivalent peptide was 2.76 (experiment 1) or 1.59 (experiment 2); by contrast, the corresponding NES from peptide-treated cells (+Nutlin) was 1.97 or 0.92, respectively. These results further support a role for the bivalent peptide in the suppression of p53 response in Nutlin-treated cells.

### Example 8: Materials and Methods

Radiolabeling of the reverse transcription primer: A reverse transcriptase (RT) primer was synthesized to complement the RNA transcript 85 bases downstream of the transcription start site. The RT primer was radiolabeled in polynucleotide kinase (PNK) buffer (70 mM Tris-HCl pH 7.6, 10 mM MgCl2, 5 mM DTT) with the addition of about 150 μCi [γ-32P]ATP, 6 U of T4 PNK and 48 ng of the RT primer in a final volume of 10 μL. The reactions were then incubated at 37° C. for 45 minutes. A glycogen mixture (10 mM Tris pH 7.5, 34 mM EDTA, 1.33 mg/mL glycogen) was then added to bring the volume to 25 μL, and the reaction was passed through a G-25 column to remove excess free [γ-32P]ATP. An additional 25 μL of TE buffer (10 mM Tris pH 7.5, 1 mM EDTA) was added. The radiolabeled primer was then stored at 4° C. until needed (up to 1 week).

Purification of PIC factors for in vitro transcription: TFIIA, TFIIB, TFIID, TFIIE, TFIIF, TFIIH, Mediator, and pol II were purified as described (Knuesel et al., 2009).

In vitro transcription: Chromatinized templates and in vitro transcription assays were generated and completed as described (Knuesel et al., 2009). Chromatinized templates and in vitro transcription assays were generated and completed as described (Knuesel et al., 2009). Briefly, each activator (GAL4-p53AD or GAL4-VP16AD) was titrated to yield maximum transcription. While the activator bound the template, the general transcription factors (GTFs) were mixed in 0.1 M HEMG (10 mM HEPES pH 7.6, 100 mM KCl, 0.1 mM EDTA, 10% glycerol, 5.5 mM MgCl2) to give approximate final concentrations of 40 nM TFIIA, 10 nM IIB, 0.8 nM TFIID, 10 nM TFIIE, 10 nM TFIIF, 0.5 nM TFIIH and 2 nM pol II. A non-limiting amount of Mediator was then diluted in a separate salts mix (10 mM HEPES pH 7.6, 100 mM KCl, 2.5% PVA, 2.5% PEG, 7.5 mM MgCl2), along with 400 U of RNAseOUT, about 300 ng PC4 and about 300 ng HMGB1. On ice, the desired concentration of peptide was then added to the Mediator mix, followed by the GTF mix at a 5:11 ratio. The GTFs, Mediator and peptide were then incubated at least 5 minutes at 30° C. Then, 15 μL of the mixture was added to each reaction. PIC assembly proceeded for 15 minutes, then transcription was initiated by adding 5 μL of a solution containing 5 mM of each NTP. After thirty minutes, reactions were stopped with the addition of 150 μL Stop Buffer (20 mM EDTA, 200 mM NaCl, 1% SDS, 100 μg/mL Proteinase K, 100 μg/mL glycogen) and incubating at 37° C. for 15 minutes. RNA was isolated with 100 μL phenol/chloroform/isoamyl alcohol (pH 7.7-8.3); 140 μL of the aqueous phase was mixed with 5 μL, 7.5 M ammonium acetate and 5 μL of twenty-fold diluted, radiolabeled (32P) Reverse Transcriptase (RT) probe and transferred to a 500 μL microfuge tube. The RNA was then precipitated by adding 375 μL, 100% cold ethanol and placing at −20° C. for at least an hour.

Primer extension: Reactions were spun down at 14K RPM for 20 minutes and the ethanol was removed. Pellets were then briefly dried (speedvac) and resuspended in 10 μL Annealing Buffer (10 mM Tris-HCl pH 7.8, 1 mM EDTA, 250 mM KCl). The resuspended RNA was then incubated in a thermocycler as follows: 85° C. for 2 minutes, cool to 58° C. at 30 sec/degree, 58° C. for 10 minutes, 57° C. for 20 minutes, 56° C. for 20 minutes, 55° C. for 10 minutes, and cool to 25° C. at 30 seconds/degree. 38 μL of RT mix (20 mM Tris-HCl pH 8.7, 10 mM MgCl2, 0.1 mg/mL actinomycin D, 330 μM of each dNTP, 5 mM DTT, 0.33 U/μL Moloney Murine Leukemia Virus (MMLV) reverse transcriptase) was then added to the annealing reactions and allowed to extend for forty-five minutes at 37° C. Reactions were then stopped and precipitated by adding 300 μL cold ethanol and placed at −20° C. for at least an hour.

Separation and visualization of transcript cDNA by denaturing polyacrylamide gel electrophoresis: The cDNA reactions were spun down at 14K RPM for 25 minutes and the ethanol was removed from the pellets. After briefly drying pellets (speedvac), cDNA was resuspended in 6 μL formamide loading buffer (75% formamide, 4 mM EDTA, 0.1 mg/mL xylene cyanol, 0.1 mg/mL bromophenol blue, 33 mM NaOH), heated for 3 minutes at 90° C. and loaded onto a denaturing polyacrylamide gel (89 mM Tris base, 89 mM boric acid, 2 mM EDTA, 7 M Urea, 6% acrylamide/bisacrylamide [19:1]). Gels were run at 35 W for about 1.5 hours, then removed on filter paper and dried for 1 hour at 80° C. Gels were then exposed on a phosphorimager screen.

Peptide Synthesis Reagents: All purchased reagents were used without further purification. Standard Fmoc-protected amino acids were purchased from Novabiochem (San Diego, Calif.). Fmoc-protected olefinic amino acids, (S)-NFmoc-2-(4′-pentenyl)alanine and (R)—N-Fmoc-2-(7′-octenyl)alanine, were purchased from Okeanos Tech Jiangsu Co., Ltd (Jiangsu, P.R. China). Rink amide resin, N,N-dimethylformamide (DMF), Nhydroxybenzotriazole (HOBt), and Grubbs Catalyst™ 1st Generation were purchased from Sigma-Aldrich (St. Louis, Mo.). Trifluoroacetic acid (TFA) and dichloroethane (DCE) were purchased from Acros Organics (Fair Lawn, N.J.). N,N,N′,N′-tetramethyl-uronium-hexafluoro-phosphate (HBTU) and diisopropylethylamine (DIEA) were purchased from AmericanBio (Natick, Mass.). Anhydrous piperazine and 6-chlorobenzotriazole-1-yloxy-tris-pyrrolidinophosphonium hexafluorophosphate (PyClocK) was purchased from EMID Millipore (Billerica, Mass.). Acetic anhydride was purchased from ThermoScientific, Pierce Biotechnology (Rockford, Ill.).

Solid Phase Peptide Synthesis: Peptides were synthesized using standard Fmoc chemistry with Rink amide resin on Biotage® Initiatior+Alstra from Biotage (Charlotte, N.C.) using microwave acceleration. Fmoc deprotections were performed using 5% piperazine with 0.1 M HOBt to reduce aspartimide formation in DMF. Coupling reactions were performed using 5 equivalents of amino acid, 4.9 equivalents of HBTU, 5 equivalents of HOBt, and 10 equivalents of DIEA in DMF at 75° C. for 5 min. Fmoc-NH-(PEGn)-COOH linkers were coupled as amino acids were. All arginine residues were double coupled at 50° C. Olefinic 55 side-chain bearing residues were coupled using 3 equivalents of amino acid, 3 equivalents of PyClocK, and 6 equivalents of DIEA and stapled for 2 hours at room temperature PyClocK. Residues following olefinic residues were double coupled using standard coupling procedures. Nterminally capped peptides were generated by treating Fmoc-deprotected resin with 100 equivalents acetic anhydride and 100 equivalents DIEA for 10 minutes at room temperature. Following synthesis, resin was washed thoroughly with alternating DMF (5 mL) and DCM (10 mL) washes before subsequent cyclizing, labeling, and cleavage.

Ring Closing Olefin Metathesis: Peptides containing olefinic amino acids were washed with DCM (3×1 min) and DCE (3×1 min) prior to cyclizing on resin using Grubbs Catalyst I (20 mol % compared to peptide, or 1 equivalent compared to resin) in DCE (4 mL) for 2 h under N2. The cyclization step was performed twice (Kim et al., 2011). The resin was then washed three times with DCM (5 mL) before washing with MeOH (5 mL×5 min) twice to shrink the resin. The resin was dried under a stream of nitrogen overnight.

Peptide Cleavage: After shrinking and drying overnight, the peptide was cleaved from the resin using a 3 mL solution of trifluoroacetic acid (TFA) (81.5%), thioanisole (5%), phenol (5%), water (5%), ethanedithiol (EDT) (2.5%) and triisopropylsilane (TIPS) (1%) for 2 hours at RT on an orbital shaker. Cleaved peptides were precipitated in diethyl ether (40 mL, chilled to −80° C.), pelleted by centrifugation, washed with additional diethyl ether (40 mL, −80° C.), pelleted, redissolved in a solution of acetonitrile (ACN) and water (15% CAN), frozen, lyophilized to dryness, and reconstituted in 1 mL dimethyl sulfoxide (DMSO) prior to purification by high-performance liquid chromatography (HPLC).

Peptide Purification by HPLC: Peptide solutions were filtered through nylon syringe filters (0.45 m pore size, 4 mm diameter, Thermo Fisher Scientific) prior to HPLC purification. Peptides were purified using an Agilent 1260 Infinity HPLC system on a reverse phase Triaryl-C18 column (YMC-Triaryl-C18, 150 mm×10 mm, 5 μm, 12 nm) (YMC America, Inc.) over H2O/ACN gradients containing 0.1% TFA. Peptides were detected at 214 nm and 280 nm. Peptide purity was verified using a Shimadzu Analytical ultraperformance liquid chromatography (UPLC) system (ES Industries, West Berlin; Shimadzu Corporation, Kyoto, Japan) and a C8 reverse phase (Sonoma C8(2), 3 μm, 100 Å, 2.1×100 mm) analytical column. Analytical samples were eluted over a gradient of 15-57 60% ACN in water containing 0.1% TFA over 15 min with detection at 214 and 280 nm.

In vitro binding assays: Starting from 180 μL HeLa nuclear extract (which contains Mediator), bivalent peptide was added (to 5 μM concentration) followed by addition of purified p53AD (residues 1-70; to 2 μM concentration). A parallel experiment lacked added bivalent peptide. Each sample was allowed to incubate, with mixing, for 2 hours at 4° C. Each sample was then incubated, with mixing, over an anti-MED1 affinity resin (to immunoprecipitate Mediator from the sample) for 90 minutes at 4° C. The resin was then washed 4 times with 20 resin volumes with 0.5M KCl HEGN (20 mM HEPES, pH 7.9; 0.1 mM EDTA, 10% glycerol, 0.1% NP-40) and once with 0.15M KCl HEGN (0.02% NP-40). Material that remained bound to the resin (i.e. Mediator) was eluted with 1M glycine, pH 2.2 and subsequently probed by western. As an alternate protocol, HeLa nuclear extract (1 mL) was first incubated over a GST SREBP affinity column, washed 5 times with 0.5M HEGN, once with 0.15M HEGN, and eluted with 30 mM glutathione buffer, as described (Ebmeier and Taatjes, 2010). This material (160 μL), which is enriched in Mediator, was then incubated with p53AD (residues 1-70; to 2 μM concentration) or GSTVP16AD (residues 411-490) in the presence or absence of bivalent peptide (5 μM) at 4° C. for 1 hour. Then each sample was incubated, with mixing, over an anti-MED1 affinity resin, washed, eluted, and probed by western as described above.

Experimental time frame for RNA-Seq experiments: In a series of experiments in SJSA cells, we initially tested whether the bivalent peptide could cause a phenotypic change. SJSA cells are unusually sensitive to Nutlin-3a (Vassilev et al., 2004) and therefore if the p53 response could be persistently blocked by the bivalent peptide, peptide-treated cells would show enhanced survival following Nutlin treatment. Starting with a 24-hour Nutlin treatment (10 μM), we observed no significant effect of the bivalent peptide: similar percentages of cell death were observed in control vs. peptide-treated populations as analyzed by CellTiter-Glo assay (Promega). Although these results could be attributed to poor cellular uptake of the bivalent peptide, we also suspected that the peptide was active in cells for only a limited time (e.g. before being secreted or degraded). We next determined that a 6-hour Nutlin treatment time was the shortest that would still trigger significant SJSA cell death within 24-48 hours. However, we obtained similar results with 6-hour (or 12-hour) Nutlin treatment times (±bivalent peptide). We then tested the prospect of RNA-Seq experiments, in hopes that gene expression changes and shorter time frames would allow an assessment of bivalent peptide effects. Here, we used HCT116 cells, which show strong transcriptional response to Nutlin (Allen et al., 2014). For RNA-Seq experiments, we needed a time frame long enough to allow accumulation of p53 target gene mRNAs but short enough to enable maximum activity of the bivalent peptide (e.g. prior to its secretion, export, and/or degradation). Using RT-qPCR assays, we confirmed that a 3-hour Nutlin treatment was a minimum amount of time to reliably detect induction of p53 target genes. In addition, parallel assays confirmed that the bivalent peptide was blocking activation of p53-target genes in HCT116 cells during this time frame. RNA-Seq data have been uploaded on GEO: GSE135870.

HCT116 cell culture: HCT116 cells were grown in McCoy's media (Gibco, 16600082) with Gibco 100× Antibiotic-Antimycotic (Fisher Sci, 15240062) penicillin-streptomycin and 10% fetal bovine serum (FBS) supplementation.

Electroporation of bivalent peptide into HCT116 cells (RNA-Seq experiment 1): Two 6-well plates (HCT116 cells) were grown to about 80% confluency. Cells were then trypsinized, washed with PBS, and resuspended in 150 μL Neon Buffer R. The cells were then split into two groups: No peptide and 10 μM peptide. The cells were then drawn into a 10 μL Neon pipet tip, electroporated and ejected into 2 mL of McCoy's 5A media without antibiotic. For each experiment, two cell electroporation aliquots were added to media containing either 0.1% DMSO (control) or 10 μM Nutlin-3a (in DMSO, to a final concentration of 0.1%). For wells containing cells electroporated with peptide, an additional 200 nM peptide was added to the well to allow for peptide uptake during the experiment. The 6-well plate was then placed back at 37° C. for 3 hours. After 3 hours, cells were scraped from the plates, transferred to a 15 mL conical vial, pelleted at 1,000×g, and washed in 10 mL phosphate buffered saline (PBS) solution. To isolate the nuclei, cells were resuspended in 10 mL lysis buffer (10 mM Tris-HCl, pH 7.4, 2 mM MgCl2, 3 mM CaCl₂), 0.5% NP-40, 10% glycerol) and thoroughly mixed. The nuclei were spun down at 1,000×g for 10 minutes, the lysis buffer was removed, and 1 mL of TRIzol was added. The nuclear RNA was isolated as described in the TRIzol instructions, except an additional phenol/chloroform extraction and chloroform-only extraction were performed to reduce contaminants. RNA was precipitated and washed twice with 75% ethanol to further remove contaminants. The RNA was then converted to cDNA using the High Capacity cDNA kit from Thermo Fisher Scientific.

Electroporation of bivalent peptide into HCT116 cells (RNA-Seq experiment 2): One 15 cm plate (HCT116 cells) was grown to about 70% confluency. Cells were then trypsinized, washed with PBS, and resuspended in 40 μL Neon Buffer R. The cells were then split into two groups: No peptide and 10 μM peptide. The cells were then drawn into a 10 μL Neon pipet tip, electroporated and ejected into 2 mL of serum-free McCoy's 5A media without antibiotic. For each experiment, two cell electroporation aliquots were added to media containing 0.1% DMSO (control) or 10 μM Nutlin-3a (in DMSO, to a final concentration of 0.1%). For wells containing cells electroporated with peptide, an additional 200 nM peptide was added to the well to allow for peptide uptake during the experiment. The 6-well plate was then placed back at 37° C. for 3 hours. After 3 hours, cells were scraped from the plate, transferred to 2 mL eppendorf tubes, pelleted at 1,000×g, and washed in 1 mL cold phosphate buffered saline (PBS) solution. To isolate the nuclei, cells were resuspended in 0.5 mL lysis buffer (10 mM Tris-HCl, pH 7.4, 2 mM MgCl2, 3 mM CaCl2), 0.5% NP-40, 10% glycerol) and mixed by pipetting up and down 20 times. The nuclei were spun down at 1,000×g for 10 minutes, the lysis buffer was removed, and 200 μL of TRIzol was added. The RNA was isolated as described in the TRIzol instructions, except an additional phenol/chloroform extraction and chloroform-only extraction were performed. RNA was precipitated and washed twice with 75% ethanol. The RNA was then converted to cDNA using the High Capacity cDNA kit from Thermo Fisher Scientific.

RNA-Seq: Quality control was performed on raw reads using FastQC (Accessed Jul. 18, 2019; bioinformatics.babraham.ac.uk/projects/fastqc/). Raw reads were then trimmed using bbduk (jgi.doe.gov/data-and-tools/bbtools/bb-tools-user-guide/bbdukguide/) with options: ktrim=r qtrim=10 k=23 mink=11 hdist=1 maq=10 minlen=25 tpe tbo literal=AAAAAAAAAAAAAAAAAAAAAAA. Trimmed reads were then mapped to the human genome (hg38) using HISAT2 (Kim et al., 2015) (options: —very-sensitive). Mapped reads over genes were counted using bedtools multibamcov and RPKM normalized. Batch correction and PCA analysis was performed in R using limma (Ritchie et al., 2015). Heatmaps were generated in Python using seaborn (seaborn.pydata.org/introduction.html) with batch corrected counts from limma. Heatmap z-scores were calculated across treatments (rows) using all data relevant to each experiment with replicates either combined or separated where appropriate. Heatmap rows and columns were clustered using Ward's method.

Linear regression analysis was performed on the core 103 p53 genes (Andrysik et al., 2017) comparing the Log 10 fold-change of the Nutlin-3a response (Nutlin vs. DMSO) against the bivalent peptide response during Nutlin-3a treatment (Peptide+Nutlin vs. Peptide+DMSO). Fitting the data to a linear regression was performed in Python using SciPy (Virtanen et al., 2019). To remove outliers, Log 10 fold-change values across either comparison were converted to z-scores and data points; a z-score greater than 3 was excluded from the regression fit and plotted outside the visible axes. Linear regression analysis was similarly performed on a random subset of 100 genes exhibiting a similar range of FPKM values as the 103 p53 core genes (Table 3). The linear regression fit was performed against H2O+DMSO expression and Peptide+DMSO expression. Outliers were handled identically to the 103 p53 core gene analysis.

Principal Component Analysis: PCA was performed using the standard prcomp function provided by the sva package for the R programming language (Leek et al., 2019). Batch effects from replicates completed on different days replicates were corrected using the removeBatchEffect function provided by the limma package (Ritchie et al., 2015) from the R programming language.

Differential Expression analysis: Differential expression analysis was performed using the DESeq2 package (Love et al., 2014) for the R programming language. Counts were generated using the utility feature counts. Initial analysis using counts across the full annotated gene showed significant skew, indicating that the baseline assumptions of the differential expression model did not hold. To correct, counts in the region from +500 of the TSS to −500 from the TES (Transcription End Site) were used to obtain suitable model weights. Those model weights were then used when performing differential expression across the full gene, which corrected the skew effect.

Gene Set Enrichment Analysis: GSEA (Subramanian et al., 2005) was performed with the Broad Institute's GSEA software on the GenePattern Server using the pre-ranked module. Normalized (DE-Seq2) (Love et al., 2014) log(2) fold-change values were used as the rank metric for all genes and compared against the Hallmark gene sets database for enrichment.

### Tables

TABLE 1
Gene expression changes
(RNA-Seq experiments 1 & 2) for 103 p53
pathway genes (Andrysik et al., 2017) for Nutlin and
Nutlin + peptide experiments.

| | Nutlin Response (log10 | Peptide Response (log10 |
|---|---|---|
| Gene | H2O_Nutlin/H2O_DMSO) | Peptide_Nutlin/H2O_Nutlin) |
| KLHL30 | 3.094622961 | −3.03257107 |
| MDM2 | 0.414256656 | −0.012842186 |
| ACER2 | 0.398437582 | −0.050373396 |
| BTG2 | 0.343079016 | −0.061486967 |
| SESN1 | 0.339274094 | −0.010539549 |
| TP53INP1 | 0.332376678 | −0.032529325 |
| GPR87 | 0.2683111 | −0.133612431 |
| FAS | 0.256363194 | −0.076645684 |
| CDKN1A | 0.244205412 | −0.040406375 |
| PHLDA3 | 0.234436281 | 0.017449999 |
| PRDM1 | 0.233294782 | 0.070166583 |
| FAM212B | 0.233207292 | −0.054676209 |
| GRIN2C | 0.221453815 | 0.009053298 |
| SESN2 | 0.198791933 | −0.039405215 |
| DDB2 | 0.198763621 | 0.004196181 |
| IKBIP | 0.185344415 | −0.064980605 |
| ANKRA2 | 0.183917436 | −0.027520857 |
| FBXO22 | 0.176141808 | −0.005866727 |
| ZNF79 | 0.173237471 | −0.012267215 |
| DRAM1 | 0.171635923 | −0.065021313 |
| PRKAB1 | 0.170941752 | −0.057770105 |
| PLK3 | 0.170540897 | −0.00613619 |
| ZNF337 | 0.170183721 | −0.06155343 |
| POLH | 0.157792561 | −0.004294926 |
| TNFRSF10B | 0.153945372 | −0.073506208 |
| DCP1B | 0.152083309 | 0.010584943 |
| ABCA12 | 0.148917112 | −0.07290909 |
| XPC | 0.146507564 | −0.027204576 |
| SUSD6 | 0.145436232 | 0.032478066 |
| DDIT4 | 0.143241274 | −0.013060704 |
| TIGAR | 0.139859784 | 0.005326025 |
| TMEM68 | 0.128969497 | −0.051896987 |
| ZMAT3 | 0.12240049 | −0.051467396 |
| RRM2B | 0.121044565 | −0.052241708 |
| GDF15 | 0.118905192 | 0.010416046 |
| PPM1D | 0.117239554 | 0.014479185 |
| GADD45A | 0.11464125 | −0.069118387 |
| SERPINE1 | 0.114240681 | −0.147210117 |
| BBC3 | 0.112408666 | 0.001322338 |
| INPP 1 | 0.106204955 | −0.031181752 |
| ETV7 | 0.104714077 | −0.165808061 |
| ASCC3 | 0.103036083 | 0.008827525 |
| RPS27L | 0.098378416 | 0.025322499 |
| PTP4A1 | 0.096223586 | −0.064582252 |
| ZNF561 | 0.093948903 | −0.056380686 |
| NADSYN1 | 0.093730387 | −0.032433397 |
| TGFA | 0.092196206 | −0.080888357 |
| CCNG1 | 0.088153039 | −0.067076471 |
| AMZ2P1 | 0.086766136 | −0.042869446 |
| ISCU | 0.086106238 | −0.015583605 |
| KANK3 | 0.085320965 | 0.035819918 |
| TSKU | 0.085014115 | −0.031875533 |
| DGKA | 0.082194315 | −0.022272145 |
| GATS | 0.082032766 | 0.018248996 |
| LIF | 0.081513298 | −0.057555707 |
| ATF3 | 0.078550081 | −0.085348865 |
| PRKX | 0.077449088 | −0.039442713 |
| BAX | 0.076025962 | 0.003918734 |
| RAP2B | 0.07513267 | −0.036753527 |
| PLK2 | 0.075127386 | −0.042996302 |
| EFNB1 | 0.073764526 | −0.067991305 |
| WDR63 | 0.069456275 | 0.012833858 |
| CD82 | 0.058602194 | −0.069193117 |
| TRIAP1 | 0.058294686 | 0.023373601 |
| BLOC1S2 | 0.05712649 | 0.011289039 |
| TMEM63B | 0.05712291 | −0.032882998 |
| TP53I3 | 0.054962448 | −0.045898562 |
| EPS8L2 | 0.049935761 | −0.01254404 |
| CSNK1G1 | 0.046487542 | −0.002156809 |
| SULF2 | 0.044703809 | −0.074238582 |
| CDC42SE1 | 0.042514261 | −0.018316659 |
| PANK1 | 0.041466464 | 0.02982482 |
| FDXR | 0.040317609 | 0.012057746 |
| ANXA4 | 0.040072206 | 0.014961391 |
| PSTPIP2 | 0.03754705 | −0.038993993 |
| RNF19B | 0.024572933 | 0.031285944 |
| AMZ2 | 0.020048981 | 0.015511566 |
| DUSP14 | 0.018559036 | −0.009640542 |
| PARD6G | 0.018328783 | 0.058196348 |
| APOBEC3H | 0.014980953 | −0.020391079 |
| FHL2 | 0.013550444 | −0.011077962 |
| CDIP1 | 0.013103744 | −0.060048037 |
| ZNF385A | 0.01251902 | 0.015690671 |
| BLCAP | 0.012436084 | −0.037178031 |
| CMBL | 0.005453436 | 0.010871636 |
| FAM210B | 0.004056782 | −0.018634477 |
| PNPO | 0.002609435 | 0.024333477 |
| ZNF561-AS1 | 0.001897421 | 0.005110387 |
| EI24 | 0.000811133 | −0.001082327 |
| CYFIP2 | −0.002735884 | 0.010550406 |
| LRP1 | −0.00278326 | 0.03753153 |
| APOBEC3C | −0.005400004 | −0.017929814 |
| TRAF4 | −0.015287758 | −0.002131986 |
| HES2 | −0.016332073 | 0.053761877 |
| CEL | −0.034284051 | 0.040630245 |
| PGF | −0.043901415 | 0.016226633 |
| PHPT1 | −0.045775965 | 0.049574979 |
| NINJ1 | −0.050519524 | 0.017233498 |
| SERTAD1 | −0.101522062 | 0.036710245 |
| ADIRF | −0.122302635 | 0.071716998 |
| NUPR1 | −0.131574314 | 0.043142025 |
| CYSRT1 | −0.135661482 | 0.148559521 |
| KLHDC7A | −2.789325153 | −0.394864943 |

TABLE 2
Summary of GSEA results for RNA-Seq experiments 1 & 2.

| | GS<br> follow | | | | NOM | FDR | FWER | RANK | |
|---|---|---|---|---|---|---|---|---|---|
| NAME | task to MSigDB | SIZE | ES | NES | p-val | q-val | p-val | AT MAX | LEADING EDGE |
| HALLMARK_OXIDATIVE_ | HALLMARK_OXIDATIVE_ | 199 | 0.36328464 | 2.4668984 | 0 | 0 | 0 | 4586 | tags = 51%, list = 18%, |
| PHOSPHORILATION [symbol] | PHOSPHORILATION | | | | | | | | signal = 62% |
| HALLMARK_MYC_TARGETS_V1 | HALLMARK_MYC_TARGETS_V1 | 198 | 0.5320982 | 2.442559 | 0 | 0 | 0 | 3988 | tags = 48%, list = 15%, |
| | | | | | | | | | signal = 53% |
| HALLMARK_UNFOLDED_PROTEIN_ | HALLMARK_UNFOLDED_PROTEIN_ | 131 | 0.34315853 | 2.2320144 | 0 | 0 | 0 | 4829 | tags = 44%, list = 25%, |
| RESPONSE | RESPONSE | | | | | | | | signal = 54% |
| HALLMARK_MFORCE_SIGNALING | HALLMARK_MFORCE_SIGNALING | 19K | 0.47743132 | 2.0936 | 0 | 0 | 0 | 3954 | tags = 38%, list = 12%, |
| | | | | | | | | | signal = 37% |
| HALLMARK_CHOLESTEROL_HOMEOSTASIS | HALLMARK_CHOLESTEROL_HOMEOSTASIS | 13 | 0.4956602 | 1.938923 | 0 | 0.004240743 | 0.001 | 4833 | tags = 43%, list = 17%, |
| | | | | | | | | | signal = 34% |
| HALLMARK_P55_PATHWAY | HALLMARK_P55_PATHWAY | 199 | 0.4394042 | 1.9615517 | 0 | 0.000288889 | 0.001 | 4437 | tags = 42%, list = 17%, |
| | | | | | | | | | signal = 38% |
| HALLMARK_DNA_REPAIR | HALLMARK_DNA_REPAIR | 143 | 0.42030445 | 1.3684753 | 0 | 0.002657438 | 0.005 | 4864 | tags = 41%, list = 18%, |
| | | | | | | | | | signal = 38% |
| HALLMARK_ESTROGEN_RESPONSE_LATE | HALLMARK_ESTROGEN_RESPONSE_LATE | 198 | 0.40325032 | 1.7435782 | 0 | 0.003048728 | 0.014 | 6373 | tags = 45%, list = 24%, |
| | | | | | | | | | signal = 39% |
| HALLMARK_APOPTOSIS | HALLMARK_APOPTOSIS | 160 | 0.41953393 | 1.7280592 | 0 | 0.003096243 | 0.008 | 4352 | tags = 36%, list = 26%, |
| | | | | | | | | | signal = 43% |
| HALLMARK_TNFA_SIGNALING_VIA_NPKB | HALLMARK_TNFA_SIGNALING_VIA_NPKB | 199 | 0.37698916 | 1.6393671 | 0 | 0.064936333 | 0.027 | 3723 | tags = 27%, list = 14%, |
| | | | | | | | | | signal = 33% |
| HALLMARK_ANDROGEN_RESPONSE | HALLMARK_ANDROGEN_RESPONSE | 141 | 0.4095722 | 1.6237841 | 0 | 0.006628383 | 0.044 | 3728 | tags = 33%, list = 14%, |
| | | | | | | | | | signal = 38% |
| HALLMARK_HYPONIA | HALLMARK_HYPONIA | 197 | 0.3733425 | 1.328537 | 0 | 0.008893735 | 0.042 | 4534 | tags = 29%, list = 37%, |
| | | | | | | | | | signal = 33% |
| HALLMARK_ADIPOGENESIS | HALLMARK_ADIPOGENESIS | 196 | 0.36337337 | 1.5863432 | 0 | 0.01027631 | 0.074 | 4880 | tags = 25%, list = 18%, |
| | | | | | | | | | signal = 43% |
| HALLMARK_INTERFERON_ALPHA_ | HALLMARK_INTERFERON_ALPHA_ | 94 | 0.4003792 | 1.5442018 | 0.006898439 | 0.033327283 | 0.108 | 5328 | tags = 31%, list = 20%, |
| RESPONSE | RESPONSE | | | | | | | | signal = 38% |
| HALLMARK_UV_RESPONSE_OP | HALLMARK_UV_RESPONSE_OP | 136 | 0.36343633 | 3.345962 | 0.003287596 | 0.013587804 | 0.204 | 5735 | tags = 38%, list = 22%, |
| | | | | | | | | | signal = 43% |
| HALLMARK_MYC_TARGETS_V2 | HALLMARK_MYC_TARGETS_V2 | 18 | 0.43200396 | 1.5324857 | 0.038058693 | 0.034583258 | 0.126 | 5390 | tags = 43%, list = 23%, |
| | | | | | | | | | signal = 55% |
| HALLMARK_FATTY_ACID_METABOLISM | HALLMARK_FATTY_ACID_METABOLISM | 158 | 0.3814864 | 1.3284763 | 0 | 0.014767687 | 0.136 | 4837 | tags = 32%, list = 13%, |
| | | | | | | | | | signal = 39% |
| HALLMARK_INTERFERON_GAMMA_ | HALLMARK_INTERFERON_GAMMA_ | 19K | 0.3506795 | 1.5316693 | 0 | 0.015424387 | 0.152 | 530K | tags = 29%, list = 26%, |
| RESPONSE | RESPONSE | | | | | | | | signal = 36% |
| HALLMARK_PEROXIDONE | HALLMARK_PEROXIDONE | 193 | 0.36193945 | 1.4588736 | 0.2134468 | 0.029486246 | 0.28 | 4983 | tags = 34%, list = 19%, |
| | | | | | | | | | signal = 42% |
| HALLMARK_NYF_TARGETS | HALLMARK_NYF_TARGETS | 199 | 0.327134 | 1.4268289 | 0.002361374 | 0.030889725 | 0.298 | 1981 | tags = 31%, list = 19%, |
| | | | | | | | | | signal = 37% |
| HALLMARK_ILD_STATS_SIGNALING | HALLMARK_ILD_STATS_SIGNALING | 199 | 0.3287353 | 1.4314392 | 0.003958624 | 0.032590758 | 0.334 | 4749 | tags = 28%, list = 18%, |
| | | | | | | | | | signal = 33% |
| HALLMARK_PGR_AKY_MTOE_SIGNALING | HALLMARK_PGR_AKY_MTOE_SIGNALING | 143 | 0.23247392 | 1.3827488 | 0.031835484 | 0.03304727 | 0.492 | 2934 | tags = 28%, list = 13%, |
| | | | | | | | | | signal = 32% |
| HALLMARK_ESTROGEN_RESPONSE_EARLY | HALLMARK_ESTROGEN_RESPONSE_EARLY | 199 | 0.31458104 | 1.3753228 | 0.007352941 | 0.038131345 | 0.42 | 2813 | tags = 35%, list = 22%, |
| | | | | | | | | | signal = 44% |
| HALLMARK_HEME_METABOLISM | HALLMARK_HEME_METABOLISM | 197 | 0.31354114 | 1.3348954 | 0.002403846 | 0.19536035 | 0.4 | 3539 | tags = 35%, list = 21%, |
| | | | | | | | | | signal = 44% |
| HALLMARK_GJM_CHECKPOINT | HALLMARK_GJM_CHECKPOINT | 200 | 0.33385863 | 1.25311855 | 0.014235984 | 0.046966300 | 0.495 | 4837 | tags = 33%, list = 13%, |
| | | | | | | | | | signal = 37% |
| HALLMARK_COMPLEMENT | HALLMARK_COMPLEMENT | 195 | 0.30326902 | 1.327083 | 0.00968523 | 0.055312887 | 0.374 | 8269 | tags = 33%, list = 34%, |
| | | | | | | | | | signal = 48% |
| HALLMARK_PROTEIN_RECESSION | HALLMARK_PROTEIN_RECESSION | 96 | 0.3204387 | 1.3967743 | 0.04264386 | 0.06376778 | 0.62 | 3088 | tags = 28%, list = 19%, |
| | | | | | | | | | signal = 37% |
| HALLMARK_GLYCOLYSIS | HALLMARK_GLYCOLYSIS | 198 | 0.10139348 | 1.3684975 | 0.01923477 | 0.06316792 | 0.838 | 4537 | tags = 28%, list = 17%, |
| | | | | | | | | | signal = 34% |
| HALLMARK_EPITHELIAL_MESENCHYMAL_ | HALLMARK_EPITHELIAL_MESENCHYMAL_ | 197 | 0.29713136 | 1.2863421 | 0.023096135 | 0.063931734 | 0.681 | 4340 | tags = 26%, list = 16%, |
| TRANSITION | TRANSITION | | | | | | | | signal = 33% |
| HALLMARK_BILE_ACID_METABOLISM | HALLMARK_BILE_ACID_METABOLISM | 112 | 0.32705882 | 1.7865184 | 0.64269863 | 0.068478326 | 0.69 | 6887 | tags = 34%, list = 23%, |
| | | | | | | | | | signal = 44% |
| HALLMARK_KENOBRYTIC_METABLISM | HALLMARK_KENOBRYTIC_METABLISM | 198 | 0.28982818 | 1.2468418 | 0.052753380 | 0.002648653 | 0.813 | 5234 | tags = 32%, list = 22%, |
| | | | | | | | | | signal = 41% |
| HALLMARK_REACTIVE_OXYGEN_SPECIES_ | HALLMARK_REACTIVE_OXYGEN_SPECIES_ | 47 | 0.35378386 | 1.2393235 | 0.00939348 | 0.13103398 | 0.362 | 4983 | tags = 34%, list = 29%, |
| PATHWAY | PATHWAY | | | | | | | | signal = 42% |
| HALLMARK_ALLOGRAPHY_REJECTION | HALLMARK_ALLOGRAPHY_REJECTION | 300 | 0.26683998 | 1.1563046 | 0.333455390 | 0.108070208 | 0.866 | 6094 | tags = 29%, list = 23%, |
| | | | | | | | | | signal = 37% |
| HALLMARK_MITOTIC_SPINDLE | HALLMARK_MITOTIC_SPINDLE | 199 | −0.33234826 | −1.3238145 | 0.037837837 | 0.28577203 | 0.763 | 6373 | tags = 38%, list = 24%, |
| | | | | | | | | | signal = 39% |
| HALLMARK_BY_RESPONSE_ON | HALLMARK_BY_RESPONSE_ON | 133 | −0.39078258 | −1.1136838 | 0.02548339 | 0.33130354 | 0.933 | 4484 | tags = 28%, list = 17%, |
| | | | | | | | | | signal = 33% |
| HALLMARK_ANGEOGENESIS | HALLMARK_ANGEOGENESIS | 36 | −0.43082583 | −1.5748369 | 0.86809389 | 0.35473284 | 0.532 | 2533 | tags = 14%, list = 13%, |
| | | | | | | | | | signal = 13% |
| HALLMARK_COAGULATION | HALLMARK_COAGULATION | 136 | 0.32333065 | 1.6591125 | 0.36863637 | 0.00107936 | 1 | 4425 | tags = 34%, list = 17%, |
| | | | | | | | | | signal = 28% |
| HALLMARK_KRAA_SIGNALING_ON | HALLMARK_KRAA_SIGNALING_ON | 194 | −0.18628363 | −1.173252 | 0.323237575 | 0.4165683 | 0.981 | 4838 | tags = 23%, list = 18%, |
| | | | | | | | | | signal = 28% |
| HALLMARK_APICAL_JUNCTION | HALLMARK_APICAL_JUNCTION | 300 | −0.289350026 | −1.205043 | 0.084308325 | 0.42830730 | 0.955 | 5594 | tags = 29%, list = 23%, |
| | | | | | | | | | signal = 36% |
| HALLMARK_TGF_BETA_SIGNALING | HALLMARK_TGF_BETA_SIGNALING | 54 | 0.38525647 | 1.0358417 | 0.45847095 | 0.4630719 | 1 | 3737 | tags = 24%, list = 30%, |
| | | | | | | | | | signal = 27% |
| HALLMARK_APICAL_SURFACE | HALLMARK_APICAL_SURFACE | 14 | −0.3272307 | −1.0699397 | 0.33435892 | 0.46539244 | 1 | 4841 | tags = 38%, list = 18%, |
| | | | | | | | | | signal = 36% |
| HALLMARK_TGR_BETA_CATENIN_ | HALLMARK_TGR_BETA_CATENIN_ | 42 | −0.35383238 | −1.0813494 | 0.31828783 | 0.3634008 | 1 | 2971 | tags = 24%, list = 13%, |
| SIGNALING | SIGNALING | | | | | | | | signal = 23% |
| HALLMARK_INFLAMMATORY_RESPONSE | HALLMARK_INFLAMMATORY_RESPONSE | 187 | 0.2781312 | 0.9817087 | 0.45685384 | 0.3054673 | 1 | 4989 | tags = 22%, list = 19%, |
| | | | | | | | | | signal = 27% |
| HALLMARK_NOTCH_SIGNALING | HALLMARK_NOTCH_SIGNALING | 32 | 0.2950075 | 0.3578951 | 0.5613026 | 0.61711114 | 1 | 3895 | tags = 25%, list = 14%, |
| | | | | | | | | | signal = 29% |
| HALLMARK_TLS_JAX_STATE_SIGNALING | HALLMARK_TLS_JAX_STATE_SIGNALING | 87 | 0.23139472 | 0.91366834 | 0.6113604 | 0.70348885 | 1 | 4528 | tags = 23%, list = 17%, |
| | | | | | | | | | signal = 28% |
| HALLMARK_HEDREPHOS_SIGNALING | HALLMARK_HEDREPHOS_SIGNALING | 35 | −0.29938886 | −0.34560295 | 0.53333336 | 0.386895435 | 1 | 3247 | tags = 24%, list = 32%, |
| | | | | | | | | | signal = 28% |
| HALLMARK_MYOGENESIS | HALLMARK_MYOGENESIS | 300 | −0.23395700 | −0.94655468 | 0.3784872 | 0.782108 | 1 | 4920 | tags = 24%, list = 29%, |
| | | | | | | | | | signal = 29% |
| HALLMARK_SPERMATOGENESIS | HALLMARK_SPERMATOGENESIS | 134 | −0.29220883 | −0.7677684 | 0.3473134 | 0.94781095 | 1 | 6545 | tags = 23%, list = 23%, |
| | | | | | | | | | signal = 33% |
| HALLMARK_PANCREAS_BETA_CELLS | HALLMARK_PANCREAS_BETA_CELLS | 40 | 0.384436334 | 0.81091364 | 0.87669494 | 0.9974943 | 1 | 10041 | tags = 18%, list = 38%, |
| | | | | | | | | | signal = 36% |
| HALLMARK_KRAS_SIGNALING_UP | HALLMARK_KRAS_SIGNALING_UP | 196 | −0.38668832 | −0.7836519 | 0.9653704 | 1 | 1 | 3632 | tags = 23%, list = 23%, |
| | | | | | | | | | signal = 28% |

[symbol]

indicates data missing or illegible when filed

TABLE 3
Gene expression changes (RNA-Seq experiments 1 & 2)
from 100 randomly selected genes with similar range of FPKM
values compared with Nutlin-induced p53 pathway genes.

| Gene | H2O_DMSO | H2O_Nutlin | Peptide_DMSO | Peptide_Nutlin |
|---|---|---|---|---|
| TUBB8 | −4.528793465 | −4.545320376 | −4.386893608 | −4.553220307 |
| SERPINB 9 | −4.60227274 | −4.552082229 | −4.578362584 | −4.583979443 |
| CYCS | −4.78100541 | −4.805921671 | −4.75071325 | −4.80671469 |
| SNORD86 | −4.879354009 | −4.935781327 | −4.902200165 | −4.932950361 |
| ACTN4 | −4.977979595 | −4.995972472 | −4.984336999 | −4.994434451 |
| FAM150B | −5.246662153 | −7.118611462 | −4.910091865 | −7.570084547 |
| GSR | −5.290823365 | −5.297025305 | −5.276305599 | −5.254966606 |
| PES1 | −5.295319804 | −5.344924155 | −5.282420088 | −5.320351555 |
| SCGB1C1 | −5.350592457 | −5.350592457 | −5.350592457 | −7.692555352 |
| NPC1 | −5.429397552 | −5.329111479 | −5.447274087 | −5.381675056 |
| HCCS | −5.46621008 | −5.509621584 | −5.459887844 | −5.516228954 |
| TM4SF5 | −5.491772513 | −5.480509714 | −2.787211894 | −8.030429596 |
| ZNF664- | −5.664834582 | −5.634852383 | −5.624652056 | −5.65933153 |
| FAM101A | | | | |
| DUS1L | −5.768444897 | −5.769014015 | −5.782849028 | −5.773608852 |
| LARS | −5.788445651 | −5.722391838 | −5.776935893 | −5.748276114 |
| TSPYL2 | −5.838844033 | −5.821953619 | −5.903188087 | −5.880994876 |
| POLR2J4 | −5.850829774 | −5.853888607 | −5.93196021 | −5.812781756 |
| OR51V1 | −5.855471242 | −8.47312736 | −5.719590889 | −8.018258208 |
| KLC1 | −5.904384927 | −5.89386423 | −5.917046712 | −5.894596071 |
| DLGAP5 | −5.925803093 | −6.013086606 | −5.925931966 | −5.975820706 |
| ME1 | −5.949523528 | −5.935182081 | −5.931373544 | −5.900767437 |
| SRF | −5.95514345 | −5.992027972 | −5.974461022 | −6.036379074 |
| CEP89 | −6.010237443 | −6.001726196 | −6.028230788 | −5.974384467 |
| LAMB1 | −6.046371337 | −5.954958068 | −6.033333947 | −6.003443823 |
| VWA5B1 | −6.048936261 | −8.89186282 | −9.186841481 | −9.157889176 |
| RNF8 | −6.05336199 | −6.043868267 | −6.056583471 | −6.039063575 |
| BUB1 | −6.058788211 | −6.065072278 | −6.029596547 | −6.054464263 |
| MAX | −6.074796182 | −6.084259347 | −6.08233461 | −6.098989748 |
| FBXO41 | −6.134887247 | −6.149740561 | −6.150155706 | −6.119868729 |
| C17orf78 | −6.177877162 | −6.190028876 | −5.962963534 | −6.193453811 |
| CFDP1 | −6.190429329 | −6.23097226 | −6.189772857 | −6.196177609 |
| BLOC1S4 | −6.35067771 | −6.255687897 | −6.293233576 | −6.274744878 |
| TMEM265 | −6.350869809 | −6.357173898 | −6.399159912 | −6.328739155 |
| GUSBP1 | −6.357147068 | −6.321609132 | −6.423360431 | −6.314468326 |
| MAST3 | −6.368954259 | −6.312781991 | −6.340683945 | −6.328216311 |
| VAV2 | −6.397874049 | −6.412763485 | −6.405211999 | −6.390750289 |
| CPE | −6.423861857 | −6.363051909 | −6.409517093 | −6.444679954 |
| ICK | −6.43689711 | −6.418633008 | −6.3981408 | −6.40117892 |
| CCL4L2 | −6.451367213 | −9.343717486 | −6.451367213 | −6.451367213 |
| GDAP1 | −6.510327582 | −6.525216346 | −6.469189139 | −6.511081986 |
| EGLN1 | −6.550619668 | −6.522574566 | −6.52851568 | −6.53425596 |
| TFB2M | −6.55138286 | −6.582552465 | −6.545312111 | −6.590295604 |
| ZNF431 | −6.559083267 | −6.468098786 | −6.576557056 | −6.506301614 |
| MIR6075 | −6.563797201 | −6.401936612 | −6.454651777 | −6.367328229 |
| CCDC167 | −6.578279779 | −6.599279855 | −6.533502498 | −6.602673079 |
| KIAA0895L | −6.616132846 | −6.571477392 | −6.658435119 | −6.576803486 |
| RAB11FIP4 | −6.634372704 | −6.651593915 | −6.641898115 | −6.621817994 |
| LRRC69 | −6.639713397 | −6.553324734 | −6.663618293 | −6.527932599 |
| MGC27345 | −6.645792204 | −6.652382849 | −6.671131102 | −6.645410314 |
| CLDN14 | −6.654897708 | −9.649013228 | −6.654897708 | −6.654897708 |
| SPINT4 | −6.722111339 | −6.722111339 | −6.106179036 | −9.340180474 |
| CH17- | −6.733599154 | −9.891974678 | −6.733599154 | −9.907886472 |
| 360D5.1 | | | | |
| AGER | −6.783723859 | −6.729925649 | −6.808754867 | −6.7236773 |
| VEGFB | −6.82326886 | −6.767166426 | −6.786229797 | −6.783107024 |
| MEG8 | −6.828278064 | −9.978453467 | −6.828278064 | −10.15675273 |
| MOB4 | −6.844771646 | −6.87687221 | −6.829213247 | −6.822052288 |
| LOC102606465 | −6.998348265 | −6.847155269 | −6.971403588 | −6.878886473 |
| TTC13 | −7.010785517 | −6.899333878 | −6.98248092 | −6.929766065 |
| QKI | −7.044028751 | −6.961989194 | −7.03241807 | −6.94827168 |
| CYP11B1 | −7.050320394 | −10.24214726 | −7.050320394 | −7.050320394 |
| CD1C | −7.079981814 | −10.28663939 | −7.079981814 | −7.079981814 |
| LIPE | −7.100494807 | −7.081970687 | −7.096510146 | −7.173035529 |
| STRC | −7.114686354 | −7.029315313 | −7.16619956 | −7.026171921 |
| C14orf177 | −7.133110039 | −10.36633172 | −7.133110039 | −7.133110039 |
| CRSP8P | −7.191710455 | −7.339940068 | −7.184064421 | −7.248073379 |
| CCDC92 | −7.20344982 | −7.176125783 | −7.160557873 | −7.14592371 |
| SCAND2P | −7.214834302 | −7.173982065 | −7.22376769 | −7.170170965 |
| DMGDH | −7.217765655 | −7.085823604 | −7.240417067 | −7.138078909 |
| MUC16 | −7.242132762 | −7.286881237 | −7.432496171 | −7.235968005 |
| XPNPEP3 | −7.284628159 | −7.249536018 | −7.297277915 | −7.266649696 |
| GCA | −7.446626957 | −7.417640663 | −7.361668398 | −7.423494709 |
| LOC400684 | −7.465267444 | −7.408520521 | −7.429005985 | −7.36791336 |
| IL6R | −7.495815291 | −7.327226608 | −7.526090816 | −7.390866444 |
| LOC101928069 | −7.592052316 | −7.582658347 | −7.541374065 | −7.601873186 |
| RRH | −7.640846508 | −7.601861323 | −7.601130267 | −7.567776845 |
| MIR4512 | −7.657871157 | −7.822193577 | −7.693452205 | −7.71944225 |
| FAM46C | −7.668486323 | −7.670635753 | −7.658979846 | −7.704448182 |
| MYLK | −7.759092676 | −7.740337509 | −7.743467758 | −7.719455718 |
| SMKR1 | −7.856346702 | −7.771469416 | −7.895498371 | −7.822681054 |
| LINC00254 | −7.923137405 | −7.868963215 | −8.55585595 | −8.246009229 |
| LOC101928673 | −7.936431014 | −7.935831356 | −8.029031105 | −7.842969052 |
| TPRXL | −7.994392629 | −7.936752973 | −7.809372893 | −7.770332024 |
| LOC100506022 | −8.071138419 | −7.894858543 | −7.895905511 | −7.717619436 |
| DARS-AS1 | −8.099221992 | −7.91267574 | −8.15362564 | −7.985471737 |
| MIR142 | −8.234018606 | −8.668546317 | −5.589974416 | −5.604577173 |
| PKD1L3 | −8.265887272 | −8.238144304 | −8.412839513 | −8.063837508 |
| OR7E47P | −8.305932568 | −7.95614793 | −8.173901859 | −7.959755908 |
| CA3 | −8.366336271 | −8.281689498 | −8.525249592 | −8.358691905 |
| FOXCUT | −8.444851373 | −5.91347311 | −5.343366954 | −7.928791126 |
| PTPRG-AS1 | −8.466723481 | −8.322690674 | −5.663240037 | −8.372993094 |
| LOC101926942 | −8.485048583 | −8.302547623 | −8.590072221 | −8.255597429 |
| LOC100507391 | −8.489121283 | −8.40467257 | −8.68724773 | −8.411692337 |
| GMFG | −8.517611181 | −9.158880439 | −8.618960874 | −8.315657561 |
| OSGEPL1-AS1 | −8.548161019 | −8.44514077 | −8.545062906 | −8.453798103 |
| CETN4P | −8.570530156 | −8.607353337 | −8.736739871 | −8.503349363 |
| ADAMTS20 | −8.592370936 | −8.818419625 | −8.61933549 | −8.243558597 |
| GDPD2 | −8.710057274 | −8.712991322 | −8.839580633 | −8.788513599 |
| ATP10A | −8.816549947 | −8.433263078 | −8.352976458 | −8.314837941 |
| ZNF208 | −8.824667432 | −5.930612044 | −8.8967028 | −9.102759144 |
| DSC1 | −9.13135981 | −6.261212541 | −5.850548658 | −8.698540856 |

### REFERENCES

All publications and patent applications cited in the specification are herein incorporated by reference in their entirety. It will be apparent to those of ordinary skill in the art that certain changes and modifications may be made thereto without departing from the spirit or scope of the appended claims.
1. Allen, M. A., Andrysik, Z., Dengler, V. L., Mellert, H. S., Guarnieri, A., Freeman, J. A., Sullivan, K. D., Galbraith, M. D., Luo, X., Kraus, W. L., et al. (2014). Global analysis of p53-regulated transcription identifies its direct targets and unexpected regulatory mechanisms. Elife (Cambridge) 3, e02200.
2. Andrysik, Z., Galbraith, M. D., Guarnieri, A. L., Zaccara, S., Sullivan, K. D., Pandey, A., MacBeth, M., Inga, A., and Espinosa, J. M. (2017). Identification of a core TP53 transcriptional program with highly distributed tumor suppressive activity. Genome research 27, 1645-1657.
3. Bernal, F., Tyler, A. F., Korsmeyer, S. J., Walensky, L. D., and Verdine, G. L. (2007). Reactivation of the p53 tumor suppressor pathway by a stapled p53 peptide. J Am Chem Soc 129, 2456-2457.
4. Bernal, F., Wade, M., Godes, M., Davis, T. N., Whitehead, D. G., Kung, A. L., Wahl, G. M., and Walensky, L. D. (2010). A stapled p53 helix overcomes HDMX-mediated suppression of p53. Cancer Cell 18, 411-422.
5. Bradner, J. E., Hnisz, D., and Young, R. A. (2017). Transcriptional Addiction in Cancer. Cell 168, 629-643.
6. Brady, C. A., Jiang, D., Mello, S. S., Johnson, T. M., Jarvis, L. A., Kozak, M. M., Broz, D. K., Basak, S., Park, E. J., McLaughlin, M. E., et al. (2011). Distinct p53 transcriptional programs dictate acute DNAdamage responses and tumor suppression. Cell 145, 571-583.
7. Brosh, R., and Rotter, V. (2009). When mutants gain new powers: news from the mutant p53 field. Nat Rev Cancer 9, 701-713.
8. Brown, C. J., Quah, S. T., Jong, J., Goh, A. M., Chiam, P. C., Khoo, K. H., Choong, M. L., Lee, M. A., Yurlova, L., Zolghadr, K., et al. (2013). Stapled peptides with improved potency and specificity that activate p53. ACS chemical biology 8, 506-512.
9. Bushweller, J. H. (2019). Targeting transcription factors in cancer—from undruggable to reality. Nat Rev Cancer 19, 611-624.
10. Carvajal, L. A., Neriah, D. B., Senecal, A., Benard, L., Thiruthuvanathan, V., Yatsenko, T., Narayanagari, S. R., Wheat, J. C., Todorova, T. I., Mitchell, K., et al. (2018). Dual inhibition of MDMX and MDM2 as a therapeutic strategy in leukemia. Science translational medicine 10.
11. Chang, Y. S., Graves, B., Guerlavais, V., Tovar, C., Packman, K., To, K. H., Olson, K. A., Kesavan, K., Gangurde, P., Mukherjee, A., et al. (2013). Stapled alpha-helical peptide drug development: a potent dual inhibitor of MDM2 and MDMX for p53-dependent cancer therapy. Proc Natl Acad Sci USA 110, E3445-3454.
12. Currie, S. L., Doane, J. J., Evans, K. S., Bhachech, N., Madison, B. J., Lau, D. K. W., McIntosh, L. P., Skalicky, J. J., Clark, K. A., and Graves, B. J. (2017). ETV4 and APi Transcription Factors Form Multivalent Interactions with three Sites on the MED25 Activator-Interacting Domain. Journal of molecular biology 429, 2975-2995.
13. Ferreon, J. C., Lee, C. W., Arai, M., Martinez-Yamout, M. A., Dyson, H. J., and Wright, P. E. (2009). Cooperative regulation of p53 by modulation of ternary complex formation with CBP/p300 and HDM2. Proc Natl Acad Sci USA 106, 6591-6596.
14. Flanagan, P. M., Kelleher-III., R. J., Sayre, M. H., Tschochner, H., and Kornberg, R. (1991). A Mediator required for activation of RNA polymerase II transcription in vitro. Nature 350, 436-438.
15. Fondell, J. D., Ge, H., and Roeder, R. G. (1996). Ligand induction of a transcriptionally active thyroid hormone receptor coactivator complex. Proc Natl Acad Sci USA 93, 8329-8333.
16. Freed-Pastor, W. A., and Prives, C. (2012). Mutant p53: one name, many proteins. Genes Dev 26, 1268-1286.
17. Gill, G., and Ptashne, M. (1988). Negative effect of the transcriptional activator GAL4. Nature 334, 721-724.
18. Herbig, E., Warfield, L., Fish, L., Fishburn, J., Knutson, B. A., Moorefield, B., Pacheco, D., and Hahn, S. (2010). Mechanism of Mediator recruitment by tandem Gcn4 activation domains and three Gal11 activator-binding domains. Mol Cell Biol 30, 2376-2390.
19. Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
20. Ito, M., Yuan, C., Malik, S., Gu, W., Fondell, J. D., Yamamura, S., Fu, Z., Zhang, X., Qin, J., and Roeder, R. G. (1999). Identity between TRAP and SMCC complexes indicates novel pathways for the function of nuclear receptors and diverse mammalian activators. Mol Cell 3, 361-370.
21. Jiang, D., Brady, C. A., Johnson, T. M., Lee, E. Y., Park, E. J., Scott, M. P., and Attardi, L. D. (2011). Full p53 transcriptional activation potential is dispensable for tumor suppression in diverse lineages. Proc Natl Acad Sci USA 108, 17123-17128.
22. Jimenez, G. S., Nister, M., Stommel, J. M., Beeche, M., Barcarse, E. A., Zhang, X. Q., O'Gorman, S., and Wahl, G. M. (2000). A transactivation-deficient mouse model provides insights into Trp53 regulation and function. Nat Genet 26, 37-43.
23. Johnson, T. M., Hammond, E. M., Giaccia, A., and Attardi, L. D. (2005). The p53QS transactivation-deficient mutant shows stress-specific apoptotic activity and induces embryonic lethality. Nat Genet 37, 145-152.
24. Kastenhuber, E. R., and Lowe, S. W. (2017). Putting p53 in Context. Cell 170, 1062-1078.
25. Khoo, K. H., Verma, C. S., and Lane, D. P. (2014). Drugging the p53 pathway: understanding the route to clinical efficacy. Nature reviews Drug discovery 13, 217-236.
26. Knuesel, M. T., Meyer, K. D., Bernecky, C., and Taatjes, D. J. (2009). The human CDK8 subcomplex is a molecular switch that controls Mediator co-activator function. Genes Dev 23, 439-451.
27. Krois, A. S., Ferreon, J. C., Martinez-Yamout, M. A., Dyson, H. J., and Wright, P. E. (2016). Recognition of the disordered p53 transactivation domain by the transcriptional adapter zinc finger domains of CREB-binding protein. Proc Natl Acad Sci USA 113, E1853-1862.
28. Kruiswijk, F., Labuschagne, C. F., and Vousden, K. H. (2015). p53 in survival, death and metabolic health: a lifeguard with a licence to kill. Nat Rev Mol Cell Biol 16, 393-405.
29. Lambert, S. A., Jolma, A., Campitelli, L. F., Das, P. K., Yin, Y., Albu, M., Chen, X., Taipale, J., Hughes, T. R., and Weirauch, M. T. (2018). The Human Transcription Factors. Cell 172, 650-665.
30. Lee, T. I., and Young, R. A. (2013). Transcriptional regulation and its misregulation in disease. Cell 152, 1237-1251.
31. Lin, J., Chen, J., Elenbaas, B., and Levine, A. J. (1994). Several hydrophobic amino acids in the p53 amino-terminal domain are required for transcriptional activation, binding to mdm-2 and the adenovirus 5 E1B 55-kD protein. Genes & Development 8, 1235-1246.
32. Meyer, K. D., Lin, S., Bernecky, C., Gao, Y., and Taatjes, D. J. (2010). p⁵³ activates transcription by directing structural shifts in Mediator. Nat Struct Mol Biol 17, 753-760.
33. Meyer, M. E., Gronemeyer, H., Turcotte, B., Bocquel, M. T., Tasset, D., and Chambon, P. (1989).
34. Steroid hormone receptors compete for factors that mediate their enhancer function. Cell 57, 433-442.
35. Milbradt, A. G., Kulkarni, M., Yi, T., Takeuchi, K., Sun, Z. Y., Luna, R. E., Selenko, P., Naar, A. M., and Wagner, G. (2011). Structure of the VP16 transactivator target in the Mediator. Nat Struct Mol Biol 18, 410-415.
36. Morrison, C. (2018). Constrained peptides' time to shine? Nature reviews Drug discovery 17, 531-533.
37. Naar, A. M., Beaurang, P. A., Robinson, K. M., Oliner, J. D., Avizonis, D., Scheek, S., Zwicker, J., Kadonaga, J. T., and Tjian, R. (1998). Chromatin, TAFs, and a novel multiprotein coactivator are required for synergistic activation by Spl and SREBP-la in vitro. Genes Dev 12, 3020-3031.
38. Nishikawa, J. L., Boeszoermenyi, A., Vale-Silva, L. A., Torelli, R., Posteraro, B., Sohn, Y. J., Ji, F., Gelev, V., Sanglard, D., Sanguinetti, M., et al. (2016). Inhibiting fungal multidrug resistance by disrupting an activator-Mediator interaction. Nature 530, 485-489.
39. Poss, Z. C., Ebmeier, C. C., and Taatjes, D. J. (2013). The Mediator complex and transcription regulation. Crit Rev Biochem Mol Biol 48, 575-608.
40. Quach, K., LaRochelle, J., Li, X. H., Rhoades, E., and Schepartz, A. (2018). Unique arginine array improves cytosolic localization of hydrocarbon-stapled peptides. Bioorganic & medicinal chemistry 26, 1197-1202.
41. Taatjes, D. J., Naar, A. M., Andel, F., Nogales, E., and Tjian, R. (2002). Structure, function, and activator-induced conformations of the CRSP coactivator. Science 295, 1058-1062.
42. Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
43. Vojnic, E., Mourao, A., Seizl, M., Simon, B., Wenzeck, L., Lariviere, L., Baumli, S., Baumgart, K., Meisterernst, M., Sattler, M., et al. (2011). Structure and VP16 binding of the Mediator Med25 activator interaction domain. Nat Struct Mol Biol 18, 404-409.
44. Wachter, F., Morgan, A. M., Godes, M., Mourtada, R., Bird, G. H., and Walensky, L. D. (2017). Mechanistic validation of a clinical lead stapled peptide that reactivates p53 by dual HDM2 and HDMX targeting. Oncogene 36, 2184-2190.
45. Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
46. Allen, M. A., Andrysik, Z., Dengler, V. L., Mellert, H. S., Guarnieri, A., Freeman, J. A., Sullivan, K. D., Galbraith, M. D., Luo, X., Kraus, W. L., et al. (2014). Global analysis of p53-regulated transcription identifies its direct targets and unexpected regulatory mechanisms. Elife (Cambridge) 3, e02200.
47. Andrysik, Z., Galbraith, M. D., Guarnieri, A. L., Zaccara, S., Sullivan, K. D., Pandey, A., MacBeth, M., Inga, A., and Espinosa, J. M. (2017). Identification of a core TP53 transcriptional program with highly distributed tumor suppressive activity. Genome research 27, 1645-1657.
48. Ebmeier, C. C., and Taatjes, D. J. (2010). Activator-Mediator binding regulates Mediator-cofactor interactions. Proc Natl Acad Sci USA 107, 11283-11288.
49. Kim, D., Langmead, B., and Salzberg, S. L. (2015). HISAT: a fast spliced aligner with low memory requirements. Nat Methods 12, 357-360.
50. Kim, Y. W., Grossmann, T. N., and Verdine, G. L. (2011). Synthesis of all-hydrocarbon stapled alphahelical peptides by ring-closing olefin metathesis. Nature protocols 6, 761-771.
51. Knuesel, M. T., Meyer, K. D., Bernecky, C., and Taatjes, D. J. (2009). The human CDK8 subcomplex is a molecular switch that controls Mediator co-activator function. Genes Dev 23, 439-451.
52. Leek, J. T., Johnson, W. E., Parker, H. S., Fertig, E. J., Jaffe, A. E., Storey, J. D., Zhang, Y., and Torres, L. C. (2019). SVA: Surrogate Variable Analysis. R package version 3321.
53. Love, M. I., Huber, W., and Anders, S. (2014). Moderated estimation of fold change and dispersion for RNA-seq data with DESeq2. Genome biology 15, 550.
54. Naar, A. M., Beaurang, P. A., Zhou, S., Abraham, S., Solomon, W., and Tjian, R. (1999). Composite co-activator ARC mediates chromatin-directed transcriptional activation. Nature 398, 828-832.
55. Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
56. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge based approach for interpreting genome-wide expression profiles. Proc Natl Acad Sci USA 102, 15545-15550.
57. Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
58. Virtanen, P., Gommers, R., Oliphant, T. E., Haberland, M., Reddy, T., Cournapeau, D., Burovski, E., Peterson, P., Weckesser, W., Bright, J., et al. (2019). SciPy 1.0—Fundamental algorithms for scientific computing in Python. arXiv:190710121.

SEQUENCE IDENTIFICATION
Amino Acid
P53AD1⁽¹⁴⁻²⁹⁾
Artificial
SEQ ID NO. 1
LSQETFSDLWKLLPEN
Amino Acid
BP1.2
Artificial
SEQ ID NO. 2
RSQRRFZRLWRLLXEN
Amino Acid
BP1.3
Artificial
SEQ ID NO. 3
RRRRRFZDLWKLLXEN
Amino Acid
BP1.4
Artificial
SEQ ID NO. 4
RSQERFZDLWKRLXRR
Amino Acid
BP1.5
Artificial
SEQ ID NO. 5
RRETFZDRWKRLXRN
Amino Acid
BP1.6
Artificial
SEQ ID NO. 6
LSQETFZRLWRRLXRR
Amino Acid
BP1.7
Artificial
SEQ ID NO. 7
LRRETFZDLWKRLXRR
Amino Acid
p53AD2⁽⁴⁵⁻⁵⁷⁾
Artificial
SEQ ID NO. 8
SPDDIEQWFTED
Amino Acid
AD2-1
Artificial
SEQ ID NO. 9
SPZDIEQWFXED
Amino Acid
AD2-2
Artificial
SEQ ID NO. 10
SPDZIEQWFTXD
Amino Acid
AD2-3
Artificial
SEQ ID NO. 11
LSXDDIXQWFTED
Amino Acid
AD2-4
Artificial
SEQ ID NO. 12
SPBDIXQWFTED
Amino Acid
AD2-5
Artificial
SEQ ID NO. 13
LSPDDIXQWFXED
Amino Acid
Bivalent peptide (BP1.4-p53AD2)
Artificial
SEQ ID NO. 14
RSQERFZDLWKRLXRRSPDDIEQWFTED

wherein SEQ ID NOs. 2-7, and 9-13 include a hydrocarbon staple between colored residues, and wherein Z and X is are a,a-disubstituted amino acids with olefin tethers for hydrocarbon-stapling, and B is an a,a-disubstituted amino acid with olefin tethers for hydrocarbon-stapling, according to FIG. 1D, incorporated herein by reference.

1. A bivalent peptide comprising a mimetic of a first activation domain of a DNA-binding transcription factor (TF) coupled with a second activation domain of said TF, wherein said bivalent peptide inhibits the interaction of the TF and the Mediator complex.

2. A bivalent peptide of claim 1, wherein said first activation domain comprises a mimetic of the first activation domain (AD1) of p53, and wherein said second activation domain comprises a second activation domain (AD2) of p53, wherein said bivalent peptide inhibits the interaction of p53 and the Mediator complex.

3. The bivalent peptide of claim 2, wherein the mimetic AD1 peptide comprises a hydrocarbon stapled mimetic AD1 peptide, wherein said hydrocarbon staple is positioned between residues Z and X, wherein Z and X are a,a-disubstituted amino acids with olefin tethers for hydrocarbon-stapling.

4. (canceled)

5. The bivalent peptide of claim 3, wherein said hydrocarbon stapled mimetic AD1 peptide comprises a hydrocarbon stapled mimetic AD1 peptide having an i, i+7 hydrocarbon staple at positions 20 and 27.

6. The bivalent peptide of claim 2, wherein the mimetic AD1 peptide further comprises a penta-arg motif.

7. The bivalent peptide of claim 2, wherein the mimetic AD1 peptide comprises a mimetic AD1 peptide according to amino acid sequence SEQ ID NO. 4, and wherein the AD2 peptide comprises an AD2 peptide according to amino acid sequence SEQ ID NO. 9.

8. (canceled)

9. The bivalent peptide of claim 2, wherein the AD2 peptide comprises a memetic AD2 peptide according to the amino acid sequences selected from the group consisting of: SEQ ID NO. 10-13.

10. The bivalent peptide of claim 2, wherein the mimetic AD1 peptide comprises a mimetic AD1 peptide according to the amino acid sequences selected from the group consisting of: SEQ ID NOs. 2-7.

11. The bivalent peptide of claim 2, wherein said bivalent peptide comprises a bivalent peptide according to amino acid sequence SEQ ID NO. 14.

12. The bivalent peptide of claim 2, wherein said mimetic AD1 peptide and said AD2 peptide are coupled by a linker domain.

13-83. (canceled)

84. A cell penetrating bivalent peptide comprising a mimetic of a first activation domain of a DNA-binding transcription factor (TF) coupled with a second activation domain of said TF, wherein at least one of the first and/or said second activation domains have a penta-arg motif, and wherein said bivalent peptide inhibits the interaction of the TF and the Mediator complex.

85. The cell penetrating bivalent peptide of claim 84, wherein said first activation domain comprises a mimetic of the first activation domain (AD1) of p53, and wherein said second activation domain comprises a second activation domain (AD2) of p53 and wherein the bivalent peptide competitively inhibits the interaction of p53 and the Mediator complex.

86. The cell penetrating bivalent peptide of claim 85, wherein the mimetic AD1 peptide comprises a hydrocarbon stapled mimetic AD1 peptide, wherein said hydrocarbon staple is positioned between residues Z, X and/or B, wherein Z, X and B are a,a-disubstituted amino acids with olefin tethers for hydrocarbon-stapling.

87. (canceled)

88. The cell penetrating bivalent peptide claim 86, wherein said hydrocarbon stapled mimetic AD1 peptide comprises a hydrocarbon stapled mimetic AD1 peptide having an i, i+7 hydrocarbon staple at positions 20 and 27.

89. The cell penetrating bivalent peptide claim 85, wherein the mimetic AD1 peptide comprises a mimetic AD1 peptide according to amino acid sequence SEQ ID NO. 4, and wherein the AD2 peptide comprises an AD2 peptide according to amino acid sequence SEQ ID NO. 9.

90. (canceled)

91. The cell penetrating bivalent peptide claim 85, wherein the AD2 peptide comprises a memetic AD2 peptide according to the amino acid sequences selected from the group consisting of: SEQ ID NO. 10-13.

92. The cell penetrating bivalent peptide claim 85, wherein the mimetic AD1 peptide comprises a mimetic AD1 peptide according to the amino acid sequences selected from the group consisting of: SEQ ID NOs. 2-7.

93. The cell penetrating bivalent peptide claim 85, wherein said bivalent peptide comprises a bivalent peptide according to amino acid sequence SEQ ID NO. 14.

94. The cell penetrating bivalent peptide claim 85, wherein said mimetic AD1 peptide and said AD2 peptide are coupled by a linker domain.

95-97. (canceled)

98. A bivalent peptide that competitively inhibits the interaction of p53 and the Mediator complex according to amino acid sequence SEQ ID NO. 14.


SEQUENCE LISTING (14 sequences)

SEQ ID NO: 1 - protein, 16 aa, Artificial
  p53AD1(14-29)
LSQETFSDLWKLLPEN

SEQ ID NO: 2 - protein, 16 aa, Artificial
  BP1.2
  misc_feature (14)..(14): Xaa can be any naturally occurring amino acid
RSQRRFZRLWRLLXEN

SEQ ID NO: 3 - protein, 16 aa, Artificial
  BP1.3
  misc_feature (14)..(14): Xaa can be any naturally occurring amino acid
RRRRRFZDLWKLLXEN

SEQ ID NO: 4 - protein, 16 aa, Artificial
  BP1.4
  misc_feature (14)..(14): Xaa can be any naturally occurring amino acid
RSQERFZDLWKRLXRR

SEQ ID NO: 5 - protein, 15 aa, Artificial
  BP1.5
  misc_feature (13)..(13): Xaa can be any naturally occurring amino acid
RRETFZDRWKRLXRN

SEQ ID NO: 6 - protein, 16 aa, Artificial
  BP1.6
  misc_feature (14)..(14): Xaa can be any naturally occurring amino acid
LSQETFZRLWRRLXRR

SEQ ID NO: 7 - protein, 16 aa, Artificial
  BP1.7
  misc_feature (14)..(14): Xaa can be any naturally occurring amino acid
LRRETFZDLWKRLXRR

SEQ ID NO: 8 - protein, 12 aa, Artificial
  p53AD2(45-57)
SPDDIEQWFTED

SEQ ID NO: 9 - protein, 12 aa, Artificial
  AD2-1
  misc_feature (10)..(10): Xaa can be any naturally occurring amino acid
SPZDIEQWFXED

SEQ ID NO: 10 - protein, 12 aa, Artificial
  AD2-2
  misc_feature (11)..(11): Xaa can be any naturally occurring amino acid
SPDZIEQWFTXD

SEQ ID NO: 11 - protein, 13 aa, Artificial
  AD2-3
  misc_feature (3)..(3): Xaa can be any naturally occurring amino acid
  misc_feature (7)..(7): Xaa can be any naturally occurring amino acid
LSXDDIXQWFTED

SEQ ID NO: 12 - protein, 12 aa, Artificial
  AD2-4
  misc_feature (6)..(6): Xaa can be any naturally occurring amino acid
SPBDIXQWFTED

SEQ ID NO: 13 - protein, 13 aa, Artificial
  AD2-5
  misc_feature (7)..(7): Xaa can be any naturally occurring amino acid
  misc_feature (11)..(11): Xaa can be any naturally occurring amino acid
LSPDDIXQWFXED

SEQ ID NO: 14 - protein, 28 aa, Artificial
  Bivalent peptide (BP1.4-p53AD2)
  misc_feature (14)..(14): Xaa can be any naturally occurring amino acid
RSQERFZDLWKRLXRRSPDDIEQWFTED